SEQUENCE LISTING (9 sequences)

SEQ ID NO: 1 - protein, 8 aa, synthetic construct
FEFKFEFK

SEQ ID NO: 2 - DNA, 21 bp, Synthetic construct
  source 1..21: mol_type = other DNA
tggacgccatgaaggttttct

SEQ ID NO: 3 - DNA, 21 bp, Synthetic construct
  source 1..21: mol_type = other DNA
tgggagccagattgtcatctc

SEQ ID NO: 4 - DNA, 20 bp, Synthetic construct
  source 1..20: mol_type = other DNA
tcacagtgtgtggtcaacat

SEQ ID NO: 5 - DNA, 20 bp, Synthetic construct
  source 1..20: mol_type = other DNA
agcccctttgttctaagcca

SEQ ID NO: 6 - DNA, 23 bp, Synthetic construct
  source 1..23: mol_type = other DNA
aaacaacctgaaccttccaaaga

SEQ ID NO: 7 - DNA, 22 bp, Synthetic construct
  source 1..22: mol_type = other DNA
gcaagtctcctcattgaatcca

SEQ ID NO: 8 - DNA, 23 bp, Synthetic construct
  source 1..23: mol_type = other DNA
atgatggcttattacagtggcaa

SEQ ID NO: 9 - DNA, 20 bp, Synthetic construct
  source 1..20: mol_type = other DNA
gtcggagattcgtagctgga

(19) United States
(12) Patent Application Publication
(10) Pub. No.: US 2026/0216095 A1
(43) Pub. Date: Jul. 30, 2026

(54) NANOSHEET-HYDROGEL COMPOSITES AND METHODS OF US

(71) Applicants: Rutgers, The State University of New Jersey, New Brunswick, NJ (US); Sung Kwang Medical Foundation, Seoul-si (KR)

(72) Inventors: Ki-Bum Lee, Monmouth Junction, NJ (US); Brian M. Conley, West Windsor, NJ (US); Inbo Han, Jeju-do (KR)

(73) Assignees: Rutgers, The State University of New Jersey, New Brunswick, NJ (US); Sung Kwang Medical Foundation, Seoul-si (KR)

(21) Appl. No.: 19/148,402

(22) PCT Filed: Jan. 17, 2024
(86) PCT No.: PCT/US2024/011736

§ 371 Date: Jul. 16, 2025

Related U.S. Application Data
(60) Provisional application No. 63/480,256, filed on Jan. 17, 2023.

(51) Int. Cl. A61K 9/70; A61K 9/06; A61K 38/18; A61K 47/02; A61K 49/18

(52) U.S. Cl. CPC A61K 9/7007; A61K 9/06; A61K 38/18; A61K 47/02; A61K 49/1803; A61K 49/1896

(57) ABSTRACT

Provided herein, inter alia, are hydrogel compositions, and use thereof for treatment of a tissue in a subject and monitoring the treatment.

### CROSS-REFERENCE TO RELATED APPLICATIONS

This application claims the benefit under 35 U.S.C. § 119(e) of the earlier filing date of U.S. Provisional Patent Application Ser. No. 63/480,256, filed on Jan. 17, 2023, which is hereby incorporated by reference in its entirety.

### STATEMENT REGARDING FEDERALLY FUNDED RESEARCH

This invention was made with government support under Grant No. CBET-1803517 awarded by The National Science Foundation and Grant Nos. 1R-1DC016612, 3R01DC016612-01S1 and 5R01DC016612-02S1 awarded by The National Institutes of Health. The government has certain rights in the invention.

### REFERENCE TO AN ELECTRONIC SEQUENCE LISTING

The contents of the electronic sequence listing (SeqList070439_01806.xml; Size: 8,730 bytes; and Date of Creation: Dec. 28, 2023) is herein incorporated by reference in its entirety.

### TECHNICAL FIELD

Provided herein, inter alia, are soft tissue devices including hydrogel composites, compositions, and use for treating a tissue injury.

### BACKGROUND

Tissue degeneration among complex extracellular matrix protein microenvironments, such as intervertebral disc (IVD) degeneration and knee meniscus tears, are often associated with debilitating pain and loss of mobility, thereby significantly compromising patients' quality of life. Despite the enormous socioeconomic burden caused by diseases and injuries to fibrocartilaginous tissues, the development of effective treatments for functional restoration of degenerative fibrocartilaginous tissues is still limited. This can be attributed to several critical issues in the degenerative/injured IVD microenvironments, such as limited regeneration capacity, pro-inflammatory factors, and the complex extracellular matrix (ECM) microenvironment of degenerative fibrocartilaginous tissues. Despite the enormous socioeconomic burden caused by diseases and injuries to fibrocartilaginous tissues, endogenous and exogenous stem cell therapies, for instance, have been considered a promising approach. However, their efficacy in modulating the environment of degenerative fibrocartilaginous tissues and differentiation into cartilaginous cells is typically impeded by unfavorable soluble and physical cues at the injury site.

Despite significant research efforts to find effective treatments for restoring the function of degenerated intervertebral discs (IVD), the limited ability to regenerate, the presence of pro-inflammatory factors, and the complex extracellular matrix (ECM) microenvironment of degenerated IVD remain major issues that must be addressed. Another barrier arises from the multiscale and heterogeneous ECM structures and mechanical environments that are typically associated with fibrocartilaginous tissues. These factors make it more challenging to recapitulate the structures and environment of degenerative/injured IVD.

For example, intervertebral discs, in their healthy state, are characterized by an inner circular region (nucleus pulposus, abbreviated as NP) with gel-like properties and a peripheral annulus fibrosus (AF) region with ligament-like properties. Across the two regions, there is a gradient interface composed of collagen and other ECM fibrils with different sizes and alignment that maintains the osmotic swelling pressure of the NP. When IVD degeneration occurs, homeostasis is disrupted, resulting in significant loss of NP tissue. Conventional biomaterials with static structures may thus fail to provide sufficient complexity for restoring degenerated discs, requiring novel biomaterial strategies for the functional restoration of injured discs. Lastly, pain management in fibrocartilaginous degeneration is also a formidable task, as even functional restoration of tissue does not ensure pain relief because of relatively poorly understood pain signaling.

Therefore, there is an urgent need to develop a therapeutic method to continually scavenge ROS, reduce inflammation, and promote regeneration, ultimately leading to tissue restoration and pain reduction following fibrocartilaginous tissue degeneration. To this end, a structurally complex dynamic biomaterial system with widely tunable biophysical and biochemical properties may be key to developing such a treatment.

### SUMMARY

To address the above challenges, provided herein, inter alia, are nanomaterial-based injectable peptide hydrogel composites that are hierarchically self-assembled from two-dimensional nanosheets (e.g., MnO₂ nanosheets) and one-dimensional fibril peptides, which mimic natural ECM, alleviate ROS-induced inflammation, and regenerate fibrocartilaginous tissue (FIG. 1).

The hydrogel composites may deliver biological and therapeutic agents (e.g., growth factors, pro-regenerative cytokines, or anti-inflammatory factors) to construct regenerative microenvironment for treating injury. For example, after IVD degeneration induction, the hydro-gel composite composition may be implanted or transplanted into a nucleotomy site (e.g., IVD) to increase ECM protein expression, facilitate nucleus pulposus cell differentiation, accelerate tissue regeneration, and/or decrease pain associated with IVD degeneration. Preferably, the hydrogel composite may be biocompatible, partially or completely biodegradable, and injectable.

In an aspect, provided is a hydrogel composite composition including nanosheets including manganese dioxide (MnO₂); self-assembling peptides; and a biologically active material.

In an aspect, provided is a method of treating a tissue injury in a subject. The method includes administering the hydrogel composite composition as described herein to the subject.

In an aspect, provided is a method of preparing a hydrogel composite. The method includes steps of preparing self-assembling peptides from an aqueous solution including peptide precursors by rapid mixing; preparing an admixture including (i) nanosheets including manganese dioxide (MnO₂) and (ii) the self-assembling peptides; adding a biological active material to the admixture.

In an aspect, provided is a method of monitoring treatment of a tissue injury in a subject comprising. The method includes administering the hydrogel composite composition as described herein to the subject; and detecting the Mn²⁺ released from the nanosheets.

In an aspect, provided is a kit for treating a tissue in a subject. The kit includes the hydrogel composite composition as described herein; and an applicator.

In an aspect, also provided is a contrast agent for magnetic resonance imaging (MRI) including thea hydrogel composite composition as described herein.

Other aspects of the invention are disclosed infra.

Where applicable or not specifically disclaimed, any one of the embodiments described herein are contemplated to be able to combine with any other one or more embodiments, even though the embodiments are described under different aspects of the invention.

### BRIEF DESCRIPTION OF THE DRAWINGS

FIGS. 1A-1D show that nanomaterial (MnO₂)-embedded peptide hydrogel (NEPH)s provide a biodegradable therapeutic platform to regenerate extracellular matrix during disc degeneration leading to robust tissue regeneration and functional outcomes. FIG. 1A shows that intervertebral Disc Degeneration (IVDD) is the loss of annulus fibrosus and nucleus pulposus cells driven by an inflammatory reaction which secretes ECM-degrading enzymes. FIG. 1B shows conventional biomaterials (i.e., biopolymers) may not directly address inflammation and the damaged ECM environment leading to chronic pain. FIG. 1C shows that two-dimensional manganese dioxide (2D-MnO₂) nanomaterial embedded peptide hydrogels (NEPH) have hierarchal structures to recapitulate natural fibril-like ECM on the nanoscale (i.e., fibril-like β-sheets), microscale (supramolecular assemblies), and macroscale (3D-printed hydrogels). FIG. 1D shows that NEP hydrogels loaded with growth factor differentiation 5 (GDF-5) injected into the intervertebral disc restore IVD tissue similar to that of uninjured discs while stimulating both collagen and aggrecan secretion and spatially regenerating annulus fibrosus and nucleus pulposus regions.

FIG. 2A shows that negatively charged two-dimensional (2D) manganese dioxide (MnO₂) nanosheets interact with beta-sheet-forming synthetic peptides (FEFKFEFK, SEQ ID NO: 1) through electrostatic interactions to form hybrid nanosheet-fibril structures (a) and hybrid inorganic/organic NEP hydrogels retain injectable hydrogel properties. FIG. 2B shows quantification of peptide fibrils from TEM images.

FIGS. 3A-3I show that biodegradable 2D-MnO₂ Nanosheets demonstrate tunable biophysical and biochemical properties to potentially enhance overall therapeutic effects. FIG. 3A shows that nanomaterial (2D-MnO₂)-embedded hybrid peptide hydrogel (NEPH) can provide a versatile platform to load growth factors and self-assembling peptides. FIG. 3B shows that fluorescence intensity of thioflavin T (ThT) shows octapeptide PepGel hydrogels self-assemble to form secondary beta-sheet structures. FIG. 3C shows that rheological data, derived from oscillatory shear strain, showing hydrogel elastic modulus can be tuned by varying the concentration of 2D-MnO₂ nanosheets (0.2 mg/ml, low and 0.8 mg/ml high) to render hydrogel stiffness (~1-3 kPa) comparable to natural host tissue such as the nucleus pulposus in the intervertebral disc (~6 kPa). FIG. 3D shows that peroxide detection assay where greater absorbance indicates a higher concentration of hydrogen peroxide. An increasing concentration of 2D-MnO₂ nanosheets scavenges a greater degree of peroxides. FIG. 3E shows that macroscopic images exhibiting the formation of oxygen gas bubbles during accelerated H₂O₂ decomposition. FIG. 3F shows that phase images of the degradation of 2D-MnO2 nanosheets in NEP hydrogels after the treatment of ascorbic acid. FIG. 3G shows that magnetic resonance imaging (MRI) T1 signal from ascorbic acid-treated NEP hydrogels which release T1 contrast agent Mn²⁺ upon degradation. FIG. 3H shows that fluorescence intensity of model growth factor FITC-Insulin release from NEP hydrogels with tunable concentrations of nanosheets demonstrating a more sustained release with increasing concentration. **p<0.005, ***p=0.0002, and ****p<0.0001 by ordinary one-way ANOVA with Tukey's multiple-comparisons test. n.s. means no significance. FIG. 3I shows redox-mediated MnO₂ nanosheet biodegradation rate.

FIGS. 4A-4F show that Nanomaterial-enhanced biophysical properties and growth factor delivery promotes therapeutic outcomes in vitro. FIG. 4A shows that incorporating 2D-MnO₂ nanosheets to form Nanomaterial (MnO2)-embedded peptide hydrogel (NEPH) s can confer several therapeutic advantages such as delaying proteolytic degradation of essential peptide fibrils, scavenge reactive oxygen species (i.e., H₂O₂) to suppress inflammation, and promote ECM deposition (i.e., collagen II) through growth factor delivery. FIG. 4B shows that degradation of hydrogels in the presence of a serine protease, trypsin demonstrating that increasing MnO₂ concentration delays protease digestion of peptides in NEP hydrogels (n=3, 0.8 mg/ml, 0.2 mg/ml MnO₂ concentration represent middle and right images, respectively). FIG. 4C shows that quantification of hydrogel area after proteolytic degradation via trypsin. Bolded groups represent those with 2D-MnO₂ nanosheets (0.2 and 0.8 mg/ml) while those not bolded represent PepGel (hydrogel with no nanomaterials). (n=3, *p=0.01) FIG. 4D shows that cell viability of nucleus pulposus cells incubated with NEPHs containing a range of 2D-MnO₂ nanosheet concentrations (0.1, 0.2, 0.3, 0.4 mg/ml from left to right) FIG. 4E shows that quantification of immunostaining for apoptotic marker, cleaved caspase 3 (Cas3) in human nucleus pulpous cells after being treated with NEPH-scavenged peroxide (H₂O₂) solution. MnO₂ concentrations are 0.2 mg/ml and 0.8 mg/ml from left to right (n=3, *p=0.03). FIG. 4F shows that delivery of growth factors (GDF-5) to human IVD nucleus pulposus cells and resulting gene expression of collagen II subunit A1 (COL2A1), the most abundant type of collagen in the nucleus pulposus region (n=3). The error bars are standard deviation around mean, n=3 experimental replicates for all experiments. Statistical analysis by ordinary one-way ANOVA with Tukey's multiple comparisons test. n.s. means no significance.

FIGS. 5A-5E show nanomaterial (2D-MnO₂)-embedded peptide hydrogel (NEPH) promotes cellular proliferation and regeneration of favorable extracellular matrix components such as collagen and aggrecan in a rat intervertebral disc degeneration model. FIG. 5A shows that a) an degenerated intervertebral disc model was induced by performing a nucleotomy in rats followed by immediate injection of hydrogels and b) Degradation of MnO₂ nanosheets releases Mn²⁺ ion which is an essential metal co-factor in prolidase activity. Collagen II is rich in proline and enhanced prolidase activity may promote collagen recycling and provide a more favorable ECM microenvironment for tissue regeneration. FIGS. 5B-5E show that histological score and quantification of H&E and IHC staining. Mean±SEM, (n=8), one-way ANOVA followed by Tukey's post-test. ***p<0.0001 scale bars, 500 μm.

FIGS. 6A-6D show nanomaterial (2D-MnO₂)-embedded peptide hydrogel (NEPH) s regenerated connective tissue and reduced pain markers in a rat intervertebral disc degeneration model to potentially improve functional outcomes. FIG. 6A shows that a) intervertebral disc degeneration was induced in a rat by performing a nucleotomy followed by immediate injection of hydrogel and b) ECM formation was improved. FIGS. 6B-6D show quantification of S&O staining, CGRP intensity, and T2-MRI signal intensity, all reaching similar quantification levels to normal intervertebral discs. Mean±SEM, (n=8), one-way ANOVA followed by Tukey's post-test. ***p<0.0001 scale bars, 500 μm.

FIG. 7A shows transmission electron microscopy (TEM) of NEPH composite structures. Fibril-like structures represent self-assembling peptides, while dark 2D structures represent 2D-MnO₂ nanosheets. Peptide fibril density appears to be greater in the surrounding areas of 2D-MnO₂ nanosheets. Scale bars as shown. FIG. 7B shows field emission Scanning electron microscopy (FESEM) images of lyophilized NEPH hydrogels exhibiting porous structures within MnO₂-peptide hybrid composites. Scale bars as shown.

FIG. 8A shows magnetic resonance imaging (MRI) T1 scan of manganese dichloride (MnCl₂) standard, water, ascorbic acid (degradation-inducing agent) followed by diluted/degraded (via ascorbic acid) PepGel hydrogels and NEPH 0.1-0.8 mg/ml. A brighter signal indicates a higher concentration of Mn²⁺ ions, a T1 contrast agent, from degraded 2D-MnO₂ nanosheets. Each column represents n=3 separate experiments. FIG. 8B shows quantification of MRI intensity (left) and released FITC-insulin (right). n=3 separate experiments. *p<0.05, ***p-0.002, ****p<0.0001 by ordinary one-way ANOVA with Tukey's multiple comparison's test.

FIG. 9A shows optical images of protease-induced degradation (24 hr. incubation) demonstrating that PepGels alone rapidly degrade in the presence of a serine protease including trypsin (10 mg/ml) and the incorporation of 2D-MnO₂ nanosheets protects synthetic peptide from proteolytic cleavage. FIG. 9B shows hydrogels incubated in PBS alone and without protease. Each column represents n=3 separate experiments. Scale bars are 500 um.

FIG. 10 shows cell viability of human primary nucleus pulposus cells (hNPCs) overnight after hydrogels (i.e., PepGel and NEPH of varying nanosheet concentrations) were incubated in a solution of hydrogen peroxide for five minutes and the subsequent solution was diluted and added into hNPC culture media. n=3 separate experiments, p=0.35 by ordinary one-way ANOVA with Tukey's multiple comparison's test. Outliers were removed via Grubb's outlier test, alpha=0.2. n.s. means no significance.

FIG. 11 shows ene expression analysis (qRT-PCR) of growth and differentiation factor (GDF-5, 200 ng/ml) to human IVD nucleus pulposus cells in a transwell culture system. The upper chamber contained 20 μL of hydrogel, while the lower chamber contained IVD cells. Cells were lysed and RNA was collected 24 hours after hydrogel treatment. Values are plotted as RQ vs Sample averages for multiple biological replicates and RQ min/max for 1 biological replicate. For CXCL1: LPS (n=1 biological replicate), PepGel (n=1 biological replicate), NEPH (n=3 biological replicates), NEPH-GDF5 (n=3 biological replicates). For IL-6: NT (n=1 biological replicate) LPS (n=1 biological replicate), PepGel (n=2 biological replicate), NEPH (n=2 biological replicates), NEPH-GDF5 (n=2 biological replicates). For IL-B: LPS (n=1 biological replicate), PepGel (n=2 biological replicate), NEPH (n=2 biological replicates), NEPH-GDF5 (n=2 biological replicates). For MMP-13: LPS (n=1 biological replicate), NEPH (n=2 biological replicates), NEPH-GDF5 (n=2 biological replicates). N.D.=not detected.

FIG. 12 shows representative T2-weighted MRI of the coccygeal discs six weeks after treatment of experimental groups labeled above columns of coronal view and sagittal view. (n=8).

FIG. 13 shows changes in the percentage of cells after injury and in G1 (PepGel), G2 (GDF-5), G3 (PepGel-GDF-5), G4 (NEPH), and G5 (NEPH+GDF-5) treated disc NP spaces positive for Iba1, CD86, and CD163. Immunopositivity was counted in the whole disc NP and calculated as relative to the total number of DAPI-positive cells. Mean±SEM, (n=8), one-way ANOVA followed by Tukey's post-test. ***p<0.0001 (normal vs injury), ***p<0.0001 (normal vs G5), ***p<0.0001 (G5 vs G1).

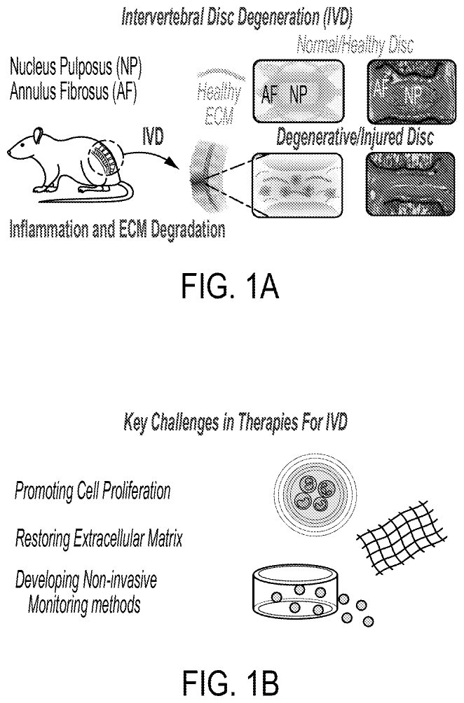

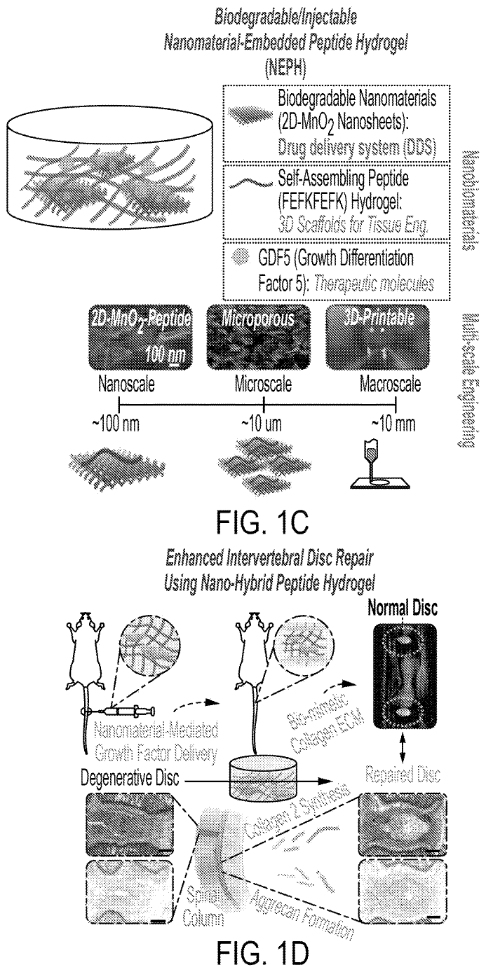

### DETAILED DESCRIPTION

Provided herein are nano-hybrid peptide hydrogels (“hydrogel composite”) that deliver pro-regenerative cytokines, suppress inflammatory factors, and construct a regenerative ECM microenvironment after IVD degeneration induction. The transplantation of this nanomaterial-based therapeutic hydrogels into a nucleotomy model increases ECM protein expression, facilitates nucleus pulposus cell differentiation, accelerates tissue regeneration, and further decreases pain associated with IVD degeneration. The nanomaterial-based hydrogels disclosed herein are highly biocompatible, completely biodegradable, and injectable. As a result, the nano-hybrid peptide hydrogels as disclosed herein provide new therapeutic strategies for the treatment of IVD degeneration as well as other degenerative diseases and injuries.

The following detailed description, given by way of example, but not intended to limit the invention solely to the specific embodiments described, may best be understood in conjunction with the accompanying drawings.

### Definitions

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, as within a range of normal tolerance in the art, for example, within 2 standard deviations of the mean. In certain embodiments, “about” can be understood as within 10%, 9%, 8%, 7%, 6%, 5%, 4%, 3%, 2%, 1%, 0.5%, 0.1%, 0.05%, or 0.01% of the stated value.

When a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

As used herein the specification, “patient,” “subject” or “subjects” or “individuals” may be used interchangeably and include, but are not limited to, mammals such as humans or non-human mammals, e.g., domesticated, agricultural or wild, animals, as well as birds, and aquatic animals. In certain embodiments, the subject is a human patient, a non-human patient or an animal subjected to medical treatment.

As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

As used herein, the term “hydrogel” is a type of gel, and refers to a water-swellable matrix, consisting of a three-dimensional network of macromolecules (e.g., self-assembling peptides, hydrophilic polymers, hydrophobic polymers, blends thereof) held together by covalent or non-covalent interactions that can absorb a substantial amount of water (e.g., 50%, 60% 70%, 80%, 90%, 95%, 96%, 97%, 98%, 99% or greater than 99% per unit of non-water molecule) to form an elastic gel. In particular, the hydrogel as described herein may contain water-swellable peptides or polymers that absorbs an amount of water greater than at least 50% of its own weight, upon immersion in an aqueous medium. The matrix may be formed of any suitable synthetic or naturally occurring material (e.g., polymers, biological polyers, peptide, or fragments thereof), particularly including synthetic or naturally occurring peptides (e.g., short peptides of SEQ ID NO: 1). In certain embodiments, the hydrogel is formed of self-assembling peptides and gelation occurs due to self-assembly interactions when a concentration of the self-assembling peptides is above a certain point without involving crosslinking moieties or chemical bonds.

A term “composite” as used herein includes any association, bonding or attachments of two or more components. For example, the “nanosheet-hydrogel composite” as used herein include at least one or more nanosheet components (e.g., carbon nanosheets, metal oxide nanosheets such as MnO₂ nanosheets) and one or more hydrogel material (e.g., self-assembling peptides). In certain embodiments, the nanosheet components and the hydrogel material may be associated with or without any chemical bonding. For example, the MnO₂ nanosheets and the self-assembling peptide hydrogel material may be associated or attached via non-covalent bond interactions (e.g., hydrogen bonding and electrostatic (ionic) bonding).

The terms “nanosheet-hydrogel composite,” “hydrogel composite,” or “composite” may be interchangeably used referring to such composite including at least nanosheets (e.g., MnO₂ nanosheets) and hydrogel (e.g., self-assembling peptides).

The term “nanosheet” as used herein refers to a 2-dimensional nanosheet. The “nanosheet” or “nanosheet-type” has a planar surface and a substantially reduced thickness (e.g., nanometer scale) compared to a width or a length of the planar surface although the surface may have regular or irregular surface properties. In certain embodiments, the nanosheets include 2-dimentional MnO₂ nanosheets, for example Mn metals that have +4 oxidation state and are bound to two oxygen atoms. In certain embodiments, the nanosheets may be formed in a single layer of MnO₂ or may include multiple layers formed by stacking MnO₂ layers. In certain embodiments, the nanosheets may include multiple layers and other substance embedded therebetween, e.g., peptides, biologically active material, polymers (e.g., adhesion layer) for suitably delivery and/or maintaining stability. In certain embodiments, the nanosheet may suitably has a thickness raning from about 0.5 nm to about 500 nm, from about 0.5 nm to about 400 nm, from about 0.5 nm to about 300 nm, from about 1 nm to about 200 nm, from about 0.5 nm to about 100 nm, from about 0.5 nm to about 50 nm, from about 0.5 nm to about 40 nm, from about 0.5 nm to about 30 nm, from about 0.5 nm to about 20 nm, from about 0.5 nm to about 10 nm, from about 0.5 nm to about 5 nm, from about 0.5 nm to about 3 nm, from about 0.5 nm to about 2 nm, from about 0.5 nm to about 1 nm, which may be controlled by the number of MnO₂ layers stacked or the amount of the intervening material.

As used herein, the term “biologically active material” refers to a material that naturally interacts or reacts with biological systems, or is engineered to interact or react with biological systems. In certain embodiments, the biologically active material may include any one of a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, and psychoactive compound, or combinations thereof. The biological system can include, for example, an individual cell, a set of cells (e.g., a cell culture), an organ, a tissue, or a multi-cellular organism.

As used herein, the term “antibody” (Ab) is used in the broadest sense, and specifically may include any immunoglobulin, whether natural, or partly, or wholly synthetically produced, including, but not limited to monoclonal antibodies, polyclonal antibodies, multispecific antibodies (for example, bispecific antibodies and polyreactive antibodies), and antibody fragments. Thus, the term “antibody,” as used in any context within this specification, is meant to include, but not be limited to, any specific binding member, immunoglobulin class and/or isotype (e.g., IgG1, IgG2a, IgG2b, IgG3, IgG4, IgM, IgAQ1, IgA2, IgD, and IgE), and biologically relevant fragment, or specific binding member thereof, including, but not limited to, Fab, F(ab′)₂, scFv (single chain or related entity) and (scFv)₂.

As used herein, the term “cytokine” may refer to any substances secreted by cells of the immune system that have an effect on other cells, including both anti-inflammatory and pro-inflammatory cytokines. Exemplary cytokines include, but are not limited to, those in the IL-1 superfamily, TNF superfamily, interferons, chemokines, and IL-6 superfamily, as well receptors of any cytokines.

As used herein, the term “nucleic acid,” may refer to a polymer composed of a multiplicity of nucleotide units (ribonucleotide, deoxyribonucleotide, or related structural variants) linked via phosphodiester bonds, including but not limited to, DNA or RNA. The term encompasses sequences that include any of the known base analogs of DNA and RNA. Examples of a nucleic acid include, and are not limited to, mRNA, miRNA, tRNA, rRNA, snRNA, siRNA, dsRNA, cDNA and DNA/RNA hybrids. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil (U), adenine (A), thymine (T), cytosine (C), guanine (G), and their derivative compounds. Nucleic acids may be obtained by chemical synthesis methods or by recombinant methods. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

As used herein, the term “peptide” may refer to peptide compounds containing two or more amino acids linked by the carboxyl group of one amino acid to the amino group of another, to form an amino acid sequence. Peptides may be purified and/or isolated from natural sources or prepared by recombinant or synthetic methods.

As used herein, the term, “self-assembling peptides,” refers to peptides that undergo spontaneous assembling into ordered nanostructures.

As used herein, the term “small molecule” may refer to non-peptidic, non-oligomeric organic compounds, either synthesized or found in nature. These compounds may be “natural product-like,” however, the term “small molecule” is not limited to “natural product-like” compounds. Small molecules are typically characterized in that they possess one or more of the following characteristics: several carbon-carbon bonds, multiple stereocenters, multiple functional groups, at least two different types of functional groups, and a molecular weight of less than 1500 Dalton, although not all, or even multiple, of these features need to be present. In certain embodiments, the “small molecule” or the like preferably has a molecule having a molecular weight of less than about 700 Dalton, e.g., less than about 1,000, 900, 800, 700, 650, 600, 550, 500, 450, 400, 350, 300, 250, 200, 100, or even 50 Dalton.

The term “growth factor” as used herein refers to a class of protein or natural substance (e.g., hormone) that stimulates growth, proliferation, maturation, healing or differentiation of cells or tissues. Exemplary growth factors may include, but not limited to, growth/differentiation factor (GDF), epidermal growth factor (EGF), platelet-derived growth factor (PDGF), vascular endothelial growth factor (VEGF), fibroblast growth factor (FGF), transforming growth factor-alpha (TGFα), nerve growth factor (NGF), placental growth factor (PIGF), VEGF/PIGF heterodimers, and connective tissue growth factor (CTGF).

In certain embodiments, the growth factor may include growth differentiation factor 5 (GDF5; e.g., UniProt ID: P43026 or P43027) which is expressed in the developing central nervous system, skeletal system, and joint. For instance, GDF5 increases survival or regenerations of neurones in the nervous system (e.g., central or peripheral nervous system).

The term “polymer” includes linear and branched polymer structures, and also encompasses crosslinked polymers as well as copolymers (which may or may not be crosslinked), thus including block copolymers, alternating copolymers, random copolymers, and the like. The polymer may be naturally occurring or obtained from synthetic sources. In certain embodiments, the polymer may be added to the composite or composition thereof to implement supplemental, desired properties to the composite.

As used herein, the term “biodegradable” refers to a material that can be broken down by biological means in a subject.

As used herein, the term “implantable” means able to be formulated for implantation via a syringe to a subject.

As used herein, the term “soft tissue” refers to tissues that connect, support, or surround other structures and organs of the body. Soft tissue includes muscles, tendons, ligaments, fascia, nerves, fibrous tissues, fat, blood vessels, and synovial membranes.

As used herein, the term “nervous system” refers to a biological system involving neuron tissues or cells and can cover a central nervous system including brain and spinal cord, and the peripheral nervous system including the autonomic and somatic nervous systems.

As used herein, the term “stable” refers to a material that does not degrade at room temperature.

As used herein, the term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. In certain embodiments, the “treating” or “treatment” includes “preventing” or “prevention” of disease. In certain embodiments, the “treating” or “treatment” may not include “preventing” or “prevention” of disease. The terms “prevent” or “preventing” refer to prophylactic and/or preventative measures, wherein the object is to prevent, or slow down the targeted pathologic condition or disorder. In addition, “treating” or “treatment” does not require complete alleviation of signs or symptoms, does not require a cure, and specifically includes protocols that have only a marginal effect on the patient.

### Nanosheets-Hydrogel Composites

In an aspect, provided is a hydrogel composite composition that includes nanosheets including manganese dioxide (MnO₂); self-assembling peptides; and a biologically active material.

The nanosheets may include a single layer of MnO₂ nanosheet or a multiple layers thereof. Each MnO₂ nanosheet may suitably have a thickness of about 0.5 to 1.5 nm, or around 1 nm. A number of the stacked MnO₂ nanosheets may be controlled to form a desired thickness of the nanosheets. Also, the nanosheets may include MnO₂ nanosheets with or without additional materials disposed therebetween.

The mean size of the nanosheets ranges from about 10 nm to about 1,000 nm, from about 10 nm to about 500 nm, from about 10 nm to about 400 nm, from about 10 nm to about 300 nm, from about 10 nm to about 200 nm, or from about 10 nm to about 100 nm, as measured at the longest dimension of the nanosheets. Preferred mean size of a single nanosheet may range from about 50 to about 100 nm, or about 100 nm. The nanosheets may be stacked in staggered way to optimize inner space between the stacked nanosheets for accommodate therapeutic or biological agents (e.g., biologically active materials) and hydrogel components (e.g., self-assembling peptide).

In certain embodiments, the mean size of a single layer MnO₂ nanosheet may range from about 10 nm to about 1,000 nm, from about 10 nm to about 500 nm, from about 10 nm to about 400 nm, from about 10 nm to about 300 nm, from about 10 nm to about 200 nm, or from about 10 nm to 100 nm. Preferred mean size of the nanosheets may range from about 50 to about 100 nm, or about 100 nm.

The nanosheets as used in the hydrogel composite may be biodegradable. The rate of degradation of the MnO₂ nanosheets may be controlled by other means, such as for example, controlling the thickness of each MnO₂ layer, the aspect ratio of the total thickness of the nanosheets to surface area of entire nanosheets, presence of additional layers (e.g., adhesion or ionomer layer), presence of other biological material (e.g., spacer proteins, extracellular matrix protein or biodegradable protein), contents of chemical components (e.g., salts, oxidants, reductants, or solvent components), and/or porosity. For example, reducing the thickness of the nanosheets by 5 times can increase the degradation speed by about 3 times; increasing the aspect ratio slows down the degradation speed by over 10 times; and increasing the protein concentrations utilized to assemble nanosheets lead to a significant increase around 7 times.

The rate of the nanosheets may affect the release of the biological or therapeutic agents (biologically active material) embedded in the nanosheets. For example, the rate at which the therapeutic agent or cells are released from the biodegradable nanoscaffolding is typically substantially equivalent to the rate at which the biodegradable scaffolding material is degraded in vivo. For example, the MnO₂ nanosheets may be controlled to degrade rapidly, with full degradation in one or more days (e.g., 2 to 3 days), or slowly, with around 20% degradation after 2 weeks. In certain embodiments, the degradation rate of the nanosheets may be be used for rationally guided drug selection and scaffold design). For example, when the nanosheets are used in the hydrogel composition for treating spinal cord injury (SCI) or intervertebral discs (IVD), slow degradation rate is preferred.

The nanosheets may be associated with the self-assembling peptides that implement hydrogel property. The self-assembling peptides are gellated when a concentration of them reaches to a specific concentration, or its ratio to the nanogel reaches to a specific limit. The self-assembling peptides may have one or more repeating amino acid residues providing hydrophobic-ity (e.g., phenylalanine, alanine, valine, isoleucine, leucine, etc.) repeating at every 2 to 5 residues. The self-assembling peptides may have one or more repeating amino acid residues providing hydrophilicity (e.g., lysine, glutamate, aspartate, arginine, etc.) e.g., repeating at every 2 to 5 residues. In certain embodiments, the self-assembling peptides include alternating hydrophobic (e.g., phenylalanine) and hydrophilic (e.g., lysine (K) and glutamate (E)) residues. The self-assembling peptides preferably include a peptide having an amino acid sequence of FEFKFEFK (SEQ ID NO: 1), or a salt, a hydrate or an isomer thereof.

In order to obtain suitably range of degradation rate of the hydrogel composite composition in a body (e.g., injury site of a subject), a ratio of the nanosheets to the self-assembling peptides may be suitably controlled. The ratio of the nanosheets to the self-assembling peptides ranges from 1:10 to 20:1. In certain embodiments, a weight ratio of the nanosheets to the self-assembling peptides is about 1:10, about 1:9, about 1:8, about 1:7, about 1:6, about 1:5, about 1:4, about 1:3, about 1:2, about 1:2.5, about 1:1.5 about 1:1. In certain embodiments, a weight ratio of the nanosheets to the self-assembling peptides is about 20:1; about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 2.5:1, about 2:1, about 1.5:1, or about 1:1.

The self-assembling peptides are embedded in the nanosheets, for example, in a space between the nanosheets. The self-assembling peptides may be attached or associated with the nanosheets via non-covalent bondings such as ionic intereaction, or hydrogen bonding or the like.

The biologically active material comprises a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, or combinations thereof.

The hydrogel composite composition as described herein may be advantageously utilized by containing one or more biologically active materials when applied to a subject, for example, on a body surface (e.g., a site of tissue injury).

In certain embodiments, the composite may include one or more growth factors (e.g., exogenous growth factors) for growth, proliferation, maturation, healing or differentiation of cells or tissues. Exemplary growth factors may include, but not limited to, growth/differentiation factor (GDF), epidermal growth factor (EGF), platelet-derived growth factor (PDGF), vascular endothelial growth factor (VEGF), fibroblast growth factor (FGF), transforming growth factor-alpha (TGFα) and/or nerve growth factors (NGF's).

For example, the hydrogel composite including EGF/TGF may advantageously be used in the acceleration of wound healing and burns, reduction in keloid scar formation (especially for burns), skin engraftment dressings, and the treatment of chronic leg ulcers. The hydrogel composite including VEGF may promote angiogenesis (blood vessel growth) or contribute to angiogenesis both indirectly and directly by stimulating proliferation of endothelial cells at the microvessel level, causing them to migrate and to alter their generic expression. The hydrogel composite including FGF may promote or induce angiogenic in vivo and its angiogenicity is enhanced by combined use of TNF. Also, keratinocyte growth factor (KGF) may be suitably contained in the hydrogel composite for wound healing and other disorders involving epithelial cell destruction. Further, the hydrogel composite including transforming growth factors (TGF) may transform various cell lines, for example, may have the ability to grow in culture for more than a limited number of generations, growth in multiple layers rather than monolayers, and the acquisi-tion of an abnormal karyotype. In certain embodiments, the hydrogel composite may include one or more growth factors (e.g., exogenous growth factors) alone or in combination with cytokines.

Particularly, the hydrogel composite includes GDF5 as the biologically active material. The hydrogel composite may be preferably used for treating an injury in a neural tissue or a nervous system (e.g., central or peripheral nervous system) by increasing survival or regenerations of neurons. For example, the hydrogel composite including GDF5 may be suitably used in treating the subject or patient having degenerated intervertebral discs (IVD), and the subject or the patient may have been treated or be greated with neurosurgery.

A concentration of the growth factor in the hydrogel composition may range from about 0.1 to about 100 μg/ml, from about 1 to about 100 μg/ml, from about 1 to about 50 μg/ml, from about 1 to about 20 μg/ml, or from about 1 to about 10 μg/ml. In certain embodiments, the concentration of the growth factor in the hydrogel composition is about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. In certain embody-ments, the concentration of GDF5 in the composition may range from about 0.1 to about 100 μg/ml, from about 1 to about 100 μg/ml, from about 1 to about 50 μg/ml, from about 1 to about 20 g/ml, or from about 1 to about 10 μg/ml. In certain embodiments, the concentration of GDF in the composition is about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. For example, the hydrogel composite composition includes the GDF5 at the concentration of about 10 μg/ml.

Suitable biologically active materials (e.g., GDF5) loaded or incorporated into the hydrogel composite compositions may be delivered or administered (e.g, with a transdermal, topical, or subdermal) and may be used in combination with other active or medical agents. Such agents include, but are not limited to, analeptic agents, analgesic agents, anesthetic agents, antiarthritic agents, anti-infective (e.g., antibiotics, antifungal agents, and antiviral agents), antiinflammatory agents, narcotic antagonists, nicotine, and nutritional agents, (e.g., vitamins, essential amino acids and fatty acids).

The hydrogel composite composition may furter include a matrix. The matrix may include a solvent component (e.g., water, polar aprotic solvent, polar protic solvent, mixtures thereof, or aqueous solution including a pharmaceutically acceptable salt formulation). The matrix may optionally include a polymer that facilitate or promote formation of stable composition. Selection of a solvent component may depend upon the characteristics of the polymer, for example, to provide the secondary forces that stabilize interactions among the polymer, hydrogel, and the nanosheets.

The solvent component may provide a secondary force to stabilize the self-assembling peptide hydrogel via (i) coulombic or inonic interaction, resulting from attraction of fixed charges on the backbone or side chains of the peptides (e.g., lysine residues will be positively charged, while aspartic or glutamic acid residues will be negatively charged, at physiological pH); (ii) dipole-dipole, resulting from interactions of permanent dipoles—the hydrogen bond, commonly found in peptides or their fragments; and/or (iii) hydrophobic interactions, resulting from association of non-polar regions of the self-assembling peptide (e.g., hydrophobic side chain of phenylalanine) due to a low tendency of non-polar species to interact with polar water molecules.

The hydrogel composite composition may suitably have a water content greater than about 10% by weight, greater than about 20% by weight, greater than about 30% by weight, greater than about 40% by weight, greater than about 50% by weight, greater than about 60% by weight, greater than about 70% by weight, greater than about 80% by weight, or greater than about 90% by weight. Alternatively, the hydrogel composite composition may suitably have a water content of about 10 to 90% by weight, about 10 to 80% by weight, of about 10 to 70% by weight, of about 10 to 60% by weight, of about 10 to 50% by weight, of about 10 to 40% by weight, of about 20 to 80% by weight, about 20 to 60% by weight, about 40 to 80% by weight, or about 40 to 60% by weight.

The hydrogel composite compositions may also include additional, optional additive components. Such components may include, for example, permeation enhancers fillers, preservatives, pH regulators, softeners, thickeners, pigments, dyes, refractive particles, stabilizers, toughening agents, detackifiers, and pharmaceutical agents (e.g., antibiotics, angiogenesis promoters, antifungal agents, immunosuppressing agents, antibodies, and the like). These additives, and amounts thereof, are selected in such a way that they do not significantly interfere with the desired chemical and physical properties of the hydrogel composition.

For example, in topical and transdermal use of the hydrogel composites, the composition or the matrix may further include a permeation enhancer to enhance the rate of incorporation or penetration of the biologically active materials into or through the skin. Suitable enhancers include, for example, sulfoxides such as dimethylsulfoxide (DMSO) and decylmethylsulfoxide, ethers such as diethylene glycol monoethyl ether (available commercially as Transcutol) and diethylene glycol monomethyl ether, surfactants such as sodium laurate, sodium lauryl sulfate, cetyltrimethylammonium bromide, benzalkonium chloride, poloxamer (231, 182, 184), Tween (20, 40, 60, 80), lecithin, 1-n-dodecylcyclaza-cycloheptan-2-one (e.g., Azone), alcohols such as ethanol, propanol, octanol, decanol, benzyl alcohol, and the like, fatty acids such as lauric acid, oleic acid and valeric acid, fatty acid esters such as isopropyl myristate, isopropyl palmitate, methylpropionate, and ethyl oleate, polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, terpenes, alkanones, and organic acids, particularly salicylic acid and salicylates, citric acid and succinic acid, or combinations thereof.

A filler or absorbent filler may be advantageously incorporated to control the degree of hydration when the adhesive is on the skin or other body surface. Such fillers can include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, woven and non-woven paper and cotton materials. Other suitable fillers are inert, i.e., substantially non-adsorbent, and include, for example, polyethylenes, polypropylenes, polyurethane polyether amide copolymers, polyesters and polyester copolymers, nylon and rayon.

The compositions can also include one or more preservatives. Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

The compositions may also include pH regulating compounds. Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface.

The compositions may also include suitable softening agents. Suitable softeners include citric acid esters, such as triethylcitrate or acetyl triethylcitrate, tartaric acid esters such as dibutyltartrate, glycerol esters such as glycerol diacetate and glycerol triacetate; phthalic acid esters, such as dibutyl phthalate and diethyl phthalate; and/or hydrophilic surfactants, preferably hydrophilic non-ionic surfactants, such as, for example, partial fatty acid esters of sugars, polyethylene glycol fatty acid esters, polyethylene glycol fatty alcohol ethers, and polyethylene glycol sorbitan-fatty acid esters.

The compositions may also include thickening agents. Preferred thickeners herein are naturally occurring compounds or derivatives thereof, and include, by way of example: collagen; galactomannans; starches; starch derivatives and hydrolysates; cellulose derivatives such as methyl cellulose, hydroxypropylcellulose, hydroxyethyl cellulose, and hydroxypropyl methyl cellulose; colloidal silicic acids; and sugars such as lactose, saccharose, fructose and glucose. Synthetic thickeners such as polyvinyl alcohol, vinylpyrrolidone-vinylacetate-copolymers, polyethylene glycols, and polypropylene glycols may also be used.

### Methods

In an aspect, provided is a method of treating a tissue injury in a subject. The method includes administering the hydrogel composite composition as described herein to the subject.

The subject, e.g., human patient, or non-human subject, may have injury or disorder in a neural tissue or a nervous system, a cartilage, a muscle, or a bone. For example, the subject, e.g., human patient, or non-human subject, has been or is suffering from an injury in a neural tissue or a nervous system, musculoskeletal system (e.g., a muscle, or a bone), or a connective tissue (e.g., cartilage, or ligament).

In certain embodiments, the subject has been or is suffering from an injury in a neural tissue or a nervous system, e.g., central nervous system (CNS) or peripheral nervous system (PNS). For example, the injury is in brain or sinal cord. In certain embodiments, the subject has been or is suffering from degenerated intervertebral discs (IVD). In certain embodiments, the subject has been or is treated with neurosurgery.

The hydrogel composite composition is injected or implanted on or around the injured tissue of the subject. The hydrogel composite composition may be formulated for dermal, subdermal, or transdermal administration as described herein.

The hydrogel composite composition is gellated or polymerized before the administration or after the administration. The hydrogel composite composition is gellated when it includes the self-assembling peptides above a predetermined concentration. In addition, the hydrogel composite composition may be further processed by adding a polymer resin to the matrix, and the polymer resin may be curable, e.g., by crosslinking or UV curing, in order to obtain a desired consistency, viscosity, flowability or other physical properties for administration.

The method may further include administering one or more additional therapeutic agents to enhance or synergize the treatment effects.

Also provided is a method of monitoring treatment of a tissue injury in a subject. The method includes steps of: administering the hydrogel composite composition as described above to the subject; and detecting the Mn²⁺ released from the nanosheets. The method may detect the Mn²⁺ released from the nanosheets in vitro, in vivo, or ex vivo.

The subject, e.g., human patient, or non-human subject, may have injury or disorder in a neural tissue or a nervous system, a cartilage, a muscle, or a bone. For example, the subject, e.g., human patient, or non-human subject, has been or is suffering from an injury in a neural tissue or a nervous system, musculoskeletal system (e.g., a muscle, or a bone), or a connective tissue (e.g., cartilage, or ligament).

In certain embodiments, the subject has been or is suffering from an injury in a neural tissue or a nervous system, e.g., central nervous system (CNS) or peripheral nervous system (PNS). For example, the injury is in brain or spinal cord. In certain embodiments, the subject has been or is suffering from degenerated intervertebral discs (IVD). In certain embodiments, the subject has been or is treated with neurosurgery.

For the detecting, Mn²⁺ released from the nanosheets (the hydrogel composite) administered on the injury site is detected using magnetic resonance imaging (MRI). The Mn²⁺ may be detected in the subject after about 1 hour, about 2 hours, about 3 hours, about 5 hours, about 12 hours, about 24 hours, about 48 hours, or about 72 hours. The detection may be performed in vitro, in vivo, or ex vivo.

In an aspect, provided is a method of preparing a hydrogel composite. The method includes steps of preparing self-assembling peptides to form a hydrogel; preparing an admixture comprising (i) nanosheets including manganese dioxide (MnO₂) as described herein (ii) the hydrogel formed by the self-assembling peptides; and adding the biological active material as described herein to the admixture.

The self-assembling peptides may be processed, e.g., by rapid mixing, from an aqueous solution including peptide precursors. For example, the rapid mixing is performed using by vortexing and centrifuging. Alternating hydrophobic residues in the peptide precursor is essential for sle-assembling process in aqueous medium or solution. In certain embodiments, the peptide presursors may include the peptide having the SEQ ID NO: 1, which includes hydrophobic residue (phenylalanine) at predetermined interval.

In certain embodiments, a fibil protein, e.g., collagen, may be processed to form peptide precursors. For instance, the fibril protin may be processed and broken down into monomeric species and then assembled into tropocollagen, fibrils, and supramolecular assemblies. The processed peptides may suitably have a width of about 2 nm, about 3 nm, about 4 nm, about 5 nm, about 6 nm, about 7 nm, about 8 nm, about 9 nm, or about 10 nm or a length of about 50 nm, about 100 nm, about 200 nm, about 300 nm, about 400 nm, or about 500 nm.

Further, vigorous and/or repeated mixing or vortexing, and centrifugation can form homogenously distributed MnO₂ nanosheets and peptide hydrogel mixture. Preferably, the peptide precursor is fully dissolved prior to adding to MnO₂ nanosheets.

Although particular examples and uses for the hydrogel composites have been described herein, such specific uses are not meant to be limiting. The hydrogel composites can be used for any application generally used for known hydrogels, and in particular, are useful for the repair and/or regeneration of soft tissue anywhere in the subject body.

In an aspect, provided is a kit for treating a tissue in a subject as the methods described herein. The kit includes the hydrogel composite composition as described herein and an applicator.

The suitable applicator includes an injection syringe that may be attached to a needle, for example, clinically-relevant gauge needles of 25-gauge (diameter: 250 μm), 27-gauge (diameter: 210 μm), or and 30-gauge (diameter: 160 μm). For example, the hydrogel composite may be injected via a needle in a syringe subcutaneously (subdermally) to a subject.

The kit may include a vial for a dispersion solution (e.g., water, saline solution or suitable dispersion or reconstitution fluid) that may be optionally combined with the hydrogel composite composition prior to or at the time of physician's use.

The suitable applicator may also include a bellows pack for single dose delivery systems or a multidose system, or a pressurized delivery system, e.g., an aerosol spray, upon release of pressure to areas on the injury site which are otherwise difficult to reach by direct application.

Also provided is a kit including a contrast agent for magnetic resonance imaging (MRI). The contrasting agent may include the hydrogel composite composition as described herein. The kit may optionally include an applicator for administering the contrasting agent to the site of imaging.

### EXAMPLES

### Example 1: Enhanced Intervertebral Disc Repair and Regeneration Using a Nano-Hybrid Peptide Hydrogel

### Methods

Synthesis of MnO₂ Nanosheets: To synthesize MnO₂ nanosheets, a reported redox reaction-based strategy was adapted. Briefly, 10 mL 0.6M tetramethyl ammonium pentahydrate aqueous solution was mixed with 10 mL 6 wt % hydrogen peroxide aqueous solution. Next, the mixed solution was quickly injected into a 0.3M MnCl₂ solution under vigorous stirring. An hour later, the stirring speed was reduced to 300 rpm, and the reaction was continued overnight. The dark-colored product was collected by 3000 rpm centrifugation and washed with ethanol and water for 3 times each. The final product was oven-dried under 60 degrees in the air. After re-dissolve and tip-sonicate the dried samples in water, a stable MnO₂ nanosheet suspension can be obtained. The suspension can be stored at room temperature for one month without any noticeable aggregation or degradation.

Synthesis of nanomaterial (MnO₂)-embedded peptide hydrogels (NEPHs): NEPHs were synthesized by first weighing out lyophilized peptides (FEFKFEFK (SEQ ID ID NO: 1), Biomatik). Next, phosphate buffer solution (for experiments of in vitro cell culture, reactive oxygen species (ROS), and in vivo experiments) or nuclease-free water was dissolved to desired concentration (10 mg/ml-20 mg/ml) in micro-centrifuge tubes through vigorous mixing. Samples were then vortexed for 30 seconds and centrifuged for 30 seconds (1000 rpm) to fully dissolve the peptides and remove bubble formation. Steps were repeated as necessary to ensure components were fully dissolved. For nanomaterial (MnO₂)-embedded peptide hydrogels (NEPHs), MnO₂nanosheets (3 mg/ml) were added to PepGel hydrogels and vigorously mixed with PepGels, vortexed, and centrifuged as aforementioned. Additional rounds of vortexing and centrifugation may be required to distribute nanosheets homogeneously. A light brown color should be homogenous throughout the hydrogel solution.

Atomic Force Microscopy (AFM): NEPHs were synthesized according to the previously described protocol. Next, 10 μL of diluted NEPHs (5 mg/ml, 100 μg/ml) were drop-cast on mica substrates (Ted Pella) and placed in a desiccator at 23° C. (room temperature) for two hours until dried. Mica substrates were then clamped to the stage of a Park Systems Atomic Force Microscope (AFM). The non-contact mode was used to measure substrate topography in the deposited region.

Transmission Electron Microscopy (TEM): NEPHs were synthesized according to the previously described protocol. After diluting to desired concentrations, approximately 5 μL of hydrogel sample was drop-cast onto transmission electron microscopy (TEM) grids (Manufacturer, Cat #) and placed at 23° C. (room temperature) for two hours in a desiccator until fully dried. Samples were then imaged using a Philips CM12 electron microscope. TEM images of NEPH fibers were pseudo-colored using Photoshop and fiber length/width were quantified using ImageJ analysis.

Scanning Electron Microscopy (SEM): NEPHs were synthesized according to the previously described protocol; however, nuclease-free water was used to dissolve lyophilized peptide as opposed to phosphate buffer saline (PBS) to prevent salt formation post-lyophilization and to prevent sample charging under the electron beam. Samples were then para-filmed and placed at −80° C. overnight. Samples were then removed from −80° C. and lyophilized for 48 hours under vacuum. Next, samples were carefully removed from the plastic tubes and placed on SEM grids (TED Pella) covered with carbon tape, taking care so as not to deform the lyophilized hydrogels with excessive force. Lyophilized NEPHs were then placed in an ion sputter coater (Electron Microscopy Sciences 150T ES) and pumped down. Then, gold (20 nm) was sputter-coated on each sample. Afterwards, samples were loaded into a Zeiss Sigma Scanning Electron Microscope and imaged using an SE2 detector at 5 kV.

Confocal microscopy: NEPHs were synthesized according to the previously described protocol to yield a final concentration of 10 μM of thioflavin T (dissolved in water), a final peptide concentration of 20 mg/ml, and a final concentration of 0.2 mg/ml of 2D-MnO₂ nanosheets. Beforehand, 2D-MnO2 nanosheets were coated with rhodamine B (RhB) overnight. For this, 2D-MnO₂ nanosheets (3 mg/ml) were incubated in Rhodamine B (1 mM) overnight with light shaking. Afterwards, PBS was added drop-wise, and the solution was spun down at 8,000 rpm for 5 min to precipitate 2D-MnO₂ nanosheets. The coated nanosheets were washed and centrifuged with water three times and resuspended in water. Just before imaging, 20 μL of NEPH hydrogel was drop-cast on a single clean glass slide (75 mm×25 mm). Afterwards, another clean glass slide (25 mm×25 mm) was gently placed on top to prevent the microscope objective from contacting the hydrogel material and permit confocal imaging. Lastly, Z-stack images were taken with a Zeiss LSM 800 confocal microscope.

Thioflavin T (ThT) Assay: PepGel hydrogels were synthesized according to the previously described protocol. 10 μM of Thioflavin T in water was added to dissolve lyophilized peptide (FEFKFEFK, SEQ ID NO: 1) to yield PepGel hydrogels with a final peptide concentration of 20 mg/ml. Next, 20 μl of peptide hydrogels were drop-cast into 96-well plates. PBS was added (180 μl) to fully submerged the hydrogel. Then, fluorescence intensity (440 nm excitation and 482 nm emission) was measured using a Tecan Microplate Reader and normalized to PBS as a control. Control groups were directly pipetted (200 μl) into 96-well plates.

Rheometry: NEPHs were synthesized according to the previously described protocol. Next, 200 μl of hydrogel was dispensed onto the platform of a Kinexus Ultra Rotational Rheometer (Malvern Instruments). First, an amplitude sweep was performed. Then, a single strain oscillatory test was performed under a frequency of 1 Hz, 1% strain, at a 0.25 mm gap. G′ or elastic components are plotted using the values at 30-second increments.

Synthesis of GDF5-functionalized nanosheets and hybridization with peptide hydrogel: To prepare GDF5-functionalized MnO₂ nanosheets, GDF5 (Peprotech, Catalog No. 120-01) was first dissolved in ultrapure water at a concentration of 1.0 mg/mL. Next, GDF-5 (1.0 mg/ml) solution was drop-wise added into MnO₂ (3 mg/ml) nanosheet aqueous solution at approximately a 1:3 ratio (v/v) under shaking. The mixed solution was continued with shaking overnight at room temperature, then transferred into the peptide hydrogel precursor solution in PBS to yield a final concentration of 0.1 mg/ml MnO₂ nanosheets and 10 μg/ml GDF-5. Next, samples were mixed and pipetted several times to which a light-brown hybrid hydrogel was then formed. Samples were then vortexed for 30 seconds and centrifuged briefly to ensure a homogenous distribution of 2D-MnO₂ nanosheets. Control hydrogels without MnO₂ nanosheets or GDF5 peptides were also fabricated using similar procedures.

Animal Model Nucleotomy: In total, eight-weeks-old female Sprague-Dawley rats (220-240 g) were purchased from Orient Bio Inc., Korea, and were acclimatized for a week at a life/dark cycle of 12/12 h (temperature; 22±1° C. and relative humidity; 50%±1%) and free approach to food and water. The animal experiments were performed according to the direction approved by the Institutional Animal Care and Use Committee (IACUC) of CHA Bundang Medical Center (IACUC 200141). At the beginning of the surgery, the peritoneal site was sterilized with 70% alcohol. Afterward, rats were thoroughly anesthetized with a general anesthesia mixture of Zoletil® (50 mg/kg Virbac Laboratories, France) and Rompun® (10 mg/kg, Bayer, Korea) injected intraperitoneally. Then, the proximal-most part of the tail along with the pelvic area was sterilized with 70% alcohol, followed by povidone-iodine solution. A longitudinal incision of 1 cm was made along the tail to expose the lateral portion of the coccygeal disc. Subsequently, a #11 scalpel blade was inserted 1.5 mm into the coccygeal disc (Co4-5, Co5-6); then, nucleotomy was performed by disc AF incision and NP aspiration with a 22-gauge catheter on a 5-ml syringe. Nucleotomy at Co4-5 was performed to assess the effects of materials in 29 rats. Thereafter, intradiscally 10 μl injection of peptide hydrogel, GDF-5 saline solution, peptide hydrogel+GDF-5, MnO₂+peptide hydrogel and MnO₂+peptide hydrogel+GDF-5 was injected by using a 25-gauge catheter. Finally, the skin was sutured, disinfected, and an appropriate dose of analgesic (Ketoprofen, SCD Pharm. Co. Ltd., Korea) and antibiotic (Cefazolin, CKD Pharmaceuticals, Korea) for 3 days after surgery was provided. The whole surgical procedure was performed on heating pads to maintain the body temperature of rats.

Animal Experimental Design: Twenty-nine rats were randomly divided into five groups for the experiment. Group 1: peptide hydrogel, group 2: GDF-5-saline solution, group 3: peptide hydrogel+GDF-5, group 4: MnO₂+peptide hydrogel, and group 5: MnO₂+peptide hydrogel+GDF-5. Each group contains 6 animals except group 1, which contains only 5 animals. After 6 weeks of implantation, Coccygeal discs were harvested for radiologic and histologic analysis.

Cell Viability: NEPH hydrogels (20 mg/ml peptide concentration) of varying 2D-MnO₂ nanosheet concentrations (0.1, 0.2, 0.3, 0.4 mg/ml) were synthesized according to the described protocol, and 5 μl of hydrogel was pipetted into wells of a 96 well plate. Next, wells were coated with poly-l-lysine (2 μg/cm² in water) according to ScienCell manufacturer's protocol dictating overnight. Then, the wells were washed carefully with phosphate buffer saline (PBS) to remove excess PLL, which could cause cytotoxicity. Afterwards, nucleus pulposus cells purchased from Science Cell (Cat No. 4800) were thawed at 37° C. and seeded at 10,000 cells/cm² in 200 μl of nucleus pulposus cell media (ScienCell hNPC medium, Cat No. 4801) with 10% FBS, 1% Pen/strep, and 1% nucleus pulposus cell growth supplement (ScienCell, Cat Nos. 0010, 0503, and 4852 respectively). After 24 hours post-seeding, nucleus pulposus cell media was changed to remove residual DMSO, washed with PBS, and every replaced with fresh media. Media changes were performed every 2-3 days as recommended by the manufacturer. After 5 days, PrestoBlue (ThermoFisher) reagent (20 μl) was added to each well of media (200 μL) and incubated at 37° C. for 15 minutes. Afterwards, fluorescence intensity (excitation: 560 nm emission: 590 nm) was measured (iTecan Micro-plate Reader) and normalized to control (no treatment).

ROS-Induced Apoptosis: NEPH hydrogels (20 mg/ml) of varying 2D-MnO₂ nanosheet concentrations (0.2, 0.8 mg/ml) were synthesized according to the described protocol. To initiate cell culture, wells were coated with poly-l-lysine (2 μg/cm²) overnight. The next day, wells were washed several times with PBS to remove excess PLL. Nucleus pulposus cells purchased from Science Cell (Cat No. 4800) were thawed at 37° C. and seeded at 10,000 cells/cm². Nucleus pulposus cell media (ScienCell hNPC medium, Cat No. 4801) with 10% FBS, 1% Pen/strep, and 1% nucleus pulposus cell growth supplement (ScienCell, Cat Nos. 0010, 0503, and 4852 respectively) was changed 24 hours post-seeding to remove residual DMSO and every two days thereafter. After reaching approximately 70% confluency, a solution of hydrogen peroxide (H₂O₂, 0.3%) (Fischer Scientific, Cat. No. H325-500) was separately prepared. Then, 20 μl of NEPH hydrogels were mixed with 300 μl of diluted H₂O₂ solutions for approximately two minutes. For control groups, H₂O₂ was added to cell culture wells to yield both 300 μM and 500 μM concentrations for positive control groups. For experimental groups, 20 μl of hydrogel (NEPH or PepGel) was mixed with 300 μl of H₂O₂ (0.3%) solutions for approximately two minutes. Then, equal parts volume as the 500 μM positive control group of the hydrogel+H₂O₂ mixture was added to the cell culture well. After 24 hours, a PrestoBlue (ThermoFisher) assay was performed (20 μL PrestoBlue reagent for every 200 μL) as previously described, and fluorescence intensities (excitation: 560 nm emission: 590 nm) were recorded and normalized to control (no treatment) using a Tecan Microplate Reader.

3D Printing of NEP Hydrogels: 3D Printing was tested using an EnvisionTEC 3D Bioplotter Manufacturer Series printer (EnvisionTEC, Inc., Dearborn, MI). A 3D model of 10 mm W×10 mm L×1.5 mm H was designed in Sketchup (Google, Inc., Mountain View, CA) and exported as STL files to Perfactory Rapid Prototype (RP) (EnvisionTEC, Inc., Dearborn, MI) to translate the STL file into a g-code. The g-code was then exported to the 3D Bioplotter. NEP hydrogels were prepared at 10 mg/mL concentration in diH2O and loaded into disposable plastic syringe. The model was printed at RT and 0.1 bar pressure at a speed of 2.5 mm/s with a 1 mm continuous strand distance, a z-offset of 0.32 mm and no contour. The inner diameter of the nozzle was 400 μm (22 gauge).

Cleaved Caspase 3/Hoechst/Actin Staining: NEPH hydrogels (20 mg/ml) of varying 2D-MnO₂ nanosheet concentrations (0.2, 0.8 mg/ml) were synthesized according to the described protocol. To initiate cell culture, wells were coated with poly-l-lysine (2 μg/cm²) overnight. The next day, wells were washed several times with PBS to remove excess PLL. Nucleus pulposus cells purchased from Science Cell (Cat No. 4800) were thawed at 37° C. and seeded at 10,000 cells/cm². Nucleus pulposus cell media (ScienCell hNPC medium, Cat No. 4801) with 10% FBS, 1% Pen/strep, and 1% nucleus pulposus cell growth supplement (ScienCell, Cat No. 0010, 0503, and 4852 respectively) was changed 24 hours post-seeding to remove residual DMSO and every two days thereafter. Hydrogel scavenging experiments were performed as previously described in ROS-induced apoptosis methods. Briefly, after reaching approximately 70% confluency, a solution of hydrogen peroxide (H₂O₂, 0.3%) (Fischer Scientific, Cat. No. H325-500) was separately prepared. Then, 20 μl of NEPH hydrogels were mixed with 300 μl of diluted H₂O₂ solutions (0.3%) for approximately two minutes. Control groups included a concentration of 300 μM H₂O₂ and equal parts volume as the control group was used for adding the hydrogel+H₂O₂ mixture to the cell culture media. After 24 hours, cells were fixed with incubation in formalin solution neutral buffered, 10% (Sigma Aldrich: Cat No. HT501128), washed several times with PBS, and stored at 4° C. For immunostaining, PBS was aspirated, and fixed cells were incubated at 4° C. overnight in blocking buffer (X % NGS, X % Triton X-100). Then, cleaved caspase-3 primary antibody (Asp175) (source: rabbit) (Abcam, Cat #: 9661) was diluted 1:500 in antibody dilution buffer, treated to each well, and stored overnight at 4° C. Next, cells were washed several times with PBS and treated with secondary anti-rabbit Alexa Fluor 488 (ThermoFisher, Cat. No. A-11008) (1:500 dilution). After 60 minutes, cells were washed several times with PBS. For Hoechst staining, Hoechst 33342 (ThermoFisher, Cat. No. H1399) was diluted 1:1000 in PBS. Each well was then treated with the Hoechst solution for 30 minutes and then washed several times with PBS. Finally, the fluorescence intensity (excitation: 496 nm, emission: 519 nm) was measured with a Tecan Microplate Reader. Cas3/Hoechst was quantified and normalized to control. For actin staining, cells were treated with Alexa-Fluor 633 Phalloidin (ThermoFisher, Cat. No. A2284). For the immunostaining images, exposure times for Hoechst, Cas3, and Actin were 100 ms, 3 s, and 300 ms, respectively. Immunostaining image LUTs in terms of min, gamma, and max are set at 0, 1, and 200 (DAPI), 110, 1, and 730 (Cas3), and 0, 1, and 150 (Actin).

Hydrogel Degradation via Ascorbic Acid: NEPH hydrogels (20 mg/ml) of varying 2D-MnO₂ nanosheet concentrations (0.1, 0.2, and 0.4 mg/ml) were synthesized according to the described protocol, and 5 μl of hydrogel was pipetted into wells in a 96 well plate. Then, ascorbic acid (Sigma Aldrich, Cat. No. A92902) 0.1 mg/ml) was added to each well, and absorbance at 600 nm was measured using a Tecan Microplate Reader in a time-dependent manner. Absorbance measurements were taken for 2.5 hours. To calculate cumulative degradation percentage, maximum absorbance values were assigned as 0% degradation, while minimum absorbance values which plateaued were assigned as 100% degradation of MnO₂ nanosheets.

ROS-Scavenging: 2D-MnO₂ nanosheets, synthesized using previous protocols, were added to hydrogen peroxide solution from a 3 mg/ml stock solution at different concentrations (0.1, 0.2, 0.4 mg/ml) and at a ratio of 1:3 2D-MnO₂ nanosheets: hydrogen peroxide and mixed thoroughly. Simultaneously, a working reagent was prepared from Pierce Quantitative Peroxide Assay Kit (Aqueous) (ThermoFisher Cat. No. 23280) according to the manufacturer's protocol. 20 μl of sample and 200 μl of working reagent were mixed, and the assay was permitted to reach an endpoint for 15 minutes. Then, absorbance at 595 nm was measured using a Tecan Microplate Reader. Absorbance values were normalized to control (water).

Magnetic Resonance Imaging of NEPH Degradation & FITC-Insulin Release: First, FITC-Insulin (0.25 mg/ml) was purified using 3 kDa filter membranes (Amicon). Next, FITC-Insulin (0.25 mg/ml) was incubated with 2D-MnO₂ nanosheets (1:3 v/v %) overnight under light shaking. Then, F.I.-loaded NEPH hydrogels (0.1 mg/ml, 0.2 mg/ml, 0.4 mg/ml) were synthesized according to the previously described protocol. Next, 20 μl NEPH hydrogels were treated with 600 μl of ascorbic acid (Sigma Aldrich, Cat. A92902) at a concentration of 1 mg/ml to induce full nanosheet degradation in Mn²⁺ ion and water. Afterwards, the solution was mixed for 30 seconds. Then, 200 μL from each experimental group was placed in a 96 well plate, and the fluorescence intensity was measured (448 nm excitation and 525 nm emission) using a Tecan Microplate Reader from the released FITC-Insulin. Next, 180 μL of solution from each experimental group was deposited into a removable well strip for MRI analysis. Additionally, a standard curve of manganese dichloride (MnCl₂) was included as a positive control. MRI was performed using an Aspect T1 MRI. Quantification was done via Image J analysis, measuring the mean intensity of sample areas. Data points for MRI analysis were normalized to water, while data points for fluorescence release were normalized to the PepGel experimental group.

NEPH Drug Release: FITC-Insulin (0.25 mg/ml) was purified using 3 kDa filter membranes (Amicon) and loaded/mixed into NEPH hydrogels with varying 2D-MnO₂ nanosheet concentration (0.2 mg/ml, 0.4 mg/ml, and 0.8 mg/ml). Hydrogels were centrifuged briefly and lightly vortexed to remove bubble formation and ensure homogenous distribution of FITC-insulin. Simultaneously, a 1% bovine serum albumin (BSA) solution was prepared and used to coat the well surface of a 48-well plate to prevent non-specific binding of released FITC-insulin. BSA solution was incubated at 37° C. overnight and washed several times with PBS to remove excess protein. Then, 20 μl of hydrogel from each experimental group was pipetted into wells of a 48-well plate to which 1 mL of PBS was added. The well plate was then para-filmed to prevent evaporation of the solution and placed on a shaker with light shaking in a 37° C. incubator. At each time point, 100 μl of supernatant was removed from the well and transferred to a 96 well plate for plate reader analysis and fluorescence intensity measurement using 448 nm excitation and 525 nm emission.

GDF-5 Delivery to hNPCs: NEPH hydrogels were synthesized according to previous protocols. GDF-5 (Peprotech) reconstituted in water was loaded on 2D-MnO₂ nanosheets (1:3 v/v %) overnight under light shaking at 4° C. GDF-5 loaded 2D MnO₂ nanosheets were then used to from NEPHs (0.2 mg/ml, 20 mg/ml, respectively). Meanwhile, a 24-well plate was coated with poly L lysine (PLL) at 2 μg/cm². Nucleus pulposus cells purchased from Science Cell (Cat No. 4800) were thawed at 37° C. and seeded at 10,000 cells/cm². Nucleus pulposus cell media (ScienCell hNPC medium, Cat No. 4801) with 10% FBS, 1% Pen/strep, and 1% nucleus pulposus cell growth supplement (ScienCell, Cat Nos. 0010, 0503, and 4852 respectively) was changed 24 hours post-seeding to remove residual DMSO and every three days thereafter. Then, transwell cell culture inserts (Corning, Cat. No. CLS3396) for 24-well culture plates were inserted into the well, and 20 μl of NEPH hydrogel loaded with GDF-5 was pipetted into the upper chamber, followed by 100 μl of hNPC media. Cells were cultured for 24 hours and trizoled for downstream PCR analysis.

qRT-PCR for RNA expression level: Cell lysate was collected with trizol (Life Technologies) treatment for 2-3 minutes and placed at −80 degrees Celsius. Next, RNA was precipitated and extracted for RT-PCR and qPCR. mRNA was converted into cDNA using the SuperScript III First-Strand Synthesis System (Life Technologies). qPCR was performed with Power SYBR Green Master Mix using a StepOne Plus PCR instrument (Applied Biosciences). Data points are plotted as RQ vs. sample (relative RNA level) for biological replicates and RQ min/max for single replicates. Col2A1 primer sequences are as follows: Forward 5′-3′: TGGACGCCATGAAGGTTTTCT (SEQ ID NO: 2) Reverse 5′-3′: TGGGAGCCAGATTGTCATCTC (SEQ ID NO: 3). CXCL1 primer sequences is as follows: Forward 5′-3′: TCACAGTGTGTGGTCAACAT (SEQ ID NO: 4) Reverse 5′-3′: AGCCCCTTTGTTCTAAGCCA (SEQ ID NO: 5). IL-6 primer sequences is as follows: Forward 5′-3′: AAACAACCTGAACCTTCCAAAGA (SEQ ID NO: 6) Reverse 5′-3′: GCAAGTCTCCTCATTGAATCCA (SEQ ID NO: 7). IL-1B primer sequences is as follows: Forward 5′-3′: ATGATGGCTTATTACAGTGGCAA (SEQ ID NO: 8) Reverse 5′-3′: GTCGGAGATTCGTAGCTGGA (SEQ ID NO: 9).

Proteolytic Hydrogel Degradation: NEPH hydrogels were synthesized according to previous protocols. Next, bovine trypsin (Sigma Aldrich) was dissolved in PBS at 10 mg/ml. Then, NEPH hydrogels and PepGel hydrogels were pipetted into 48 well plates, submerged in trypsin solution, and incubated in a 37° C. incubator overnight. After 24 hours, the well plates were imaged using phase objectives on a Nikon Ti Series microscope. Hydrogel size was then quantified using Image J software.

Mechanical Allodynia: The Von Frey test was performed to assess mechanical allodynia induced by pain in the rats. It was performed 2 days before the surgery and on days 2, 7, 14, 21, 28, 35, and 42 days after the surgery. First, to avoid the exploratory activities of rats, they were placed individually into a six-compartment rat enclosure with wire mesh floors and lids with air holes for 20 minutes habituation period. After that, on the ventral surface of the tail, a 2-g filament was applied for a maximum of 6 s with enough force. Positive responses were judged to be behaviors that occurred immediately or within six seconds, such as flinching, licking, withdrawing, or shaking the base of the tail. However, if the animals did not show any responses to the filaments when it was applied, then it was considered as a negative response. The test was carried out five times for each animal from each group. The two independent observers who were blinded by the specimen's treatment were involved in Von Frey's analysis.

Magnetic Resonance Imaging (MRI): After six weeks of implantation, 9.4T MRI (Bruker BioSpec, USA) was performed to study the changes in the structure of the disc and degree of degeneration of the coccygeal disc, signal intensity, and presence of water content in the disc. T2-weighted imaging for the coronal plane was performed as; time to repetition (TR) of 5000 ms, time to echo (TE) of 30 ms, 150 horizontal_150 vertical matrix; field of view of 15 horizontal×15 verticals, and 0.5 mm slices with 0 mm spacing between each slice. The signal intensity and MRI index (calculated as the area of NP multiplied by average signal intensity) were calculated in order to evaluate the degree of degeneration of the coccygeal disc. The high signal intensity area in the mid-coronal plane of the T2 weighted images was considered as a region of interest (ROI), as the outline of the NP. The ROI was measured by using Image J software (the National Institutes of Health, Bethesda, MD, USA) (1). The two independent observers who were blinded by the specimen's treatment were involved in measuring the MRI index.

Safranin-O staining with histological scoring and H&E staining: Histological analysis was performed after the six weeks of implantation. Rats were euthanized, and discs from each rat were harvested for histological analysis. The discs with the adjacent vertebral body were fixed in 10% neutral buffered formalin for one week, and decalcified in Rapid Cal Immuno (BBC Biochemical, Mount Vernon, WA, USA) for 2 weeks. Afterward, tissues were processed for paraffin embedding and sectioning into coronal sections (10 μm) using a microtome (Leica). The obtained sections were dewaxed, rehydrated, and stained with Safranin-O (Sigma, USA) to analyze the quantity and distribution of proteoglycan content. Finally, sections were mounted using mounting media and scanned with an OLYMPUS C-mount camera adapter (U-TVO.63XC, Tokyo, Japan). Similarly, histological scoring was performed utilizing an extensive 8-point scale for measuring IVD based on safranin-O staining. The scoring was based on the NP cellularity (0-2), AF morphology (0-2), NP matrix (0-2) and the boundary between NP and AF (0-2), resulting in four subcategories. In this case, nondegenerative characteristics were zero, mild degenerative characteristics were represented as one, and severe degenerative change was represented as two (2). The pathologists, who were completely blinded to the sample information, analyzed all the samples for histological analysis. Furthermore, the obtained sections were dewaxed, rehydrated, and stained with H&E to evaluate the tissue morphology and proteoglycan distribution.

Immunohistochemistry: After six weeks of implantation, rats were euthanized via excess carbon dioxide inhalation and coccygeal discs were collected, and immunohistochemical analysis was performed for aggrecan (disc matrix component), collagen type II (a component of disc NP matrix), CGRP (pain-marker), Iba1 (pan-macrophage), CD86 (M1-macrophage) and CD163(M2-macrophage). The harvested tissues were fixed overnight in a 4% paraformaldehyde (PFA) solution and decalcified using a decalcification solution; RapidCal Immuno (BBC Biochemical, Mount Vernon, WA, USA) for 2 weeks. Then, discs were embedded within paraffin wax and sectioned longitudinally using a microtome (Leica) into sections of 5-10 μm thickness. For the immunohistochemical staining, the first sections were dewaxed, rehydrated, and after that, stained with primary antibodies against aggrecan (1:1000, Abcam, UK), collagen type II (1:100, Abcam, UK) CGRP (1:200, Abcam, UK), Iba-1 (1:200, Abcam, UK), CD86 (1:200, Abcam, UK) and CD163 (1:200, Abcam, UK). Then, after 24 hours of incubation, sections were washed with phosphate-buffered saline with Tween 20 and again incubated with the secondary antibody anti-Rb horseradish peroxidase (Roche Diagnostics Ltd., Switzerland), and Alexa Fluor 488, 568, and 647-conjugated secondary antibodies (1:400, Invitrogen, USA). After that, specimens were carried out for the washing step; then the specimens were counter-stained with DAPI (1:500, Abcam, UK) and incubated for 10 minutes. The sections were mounted and finally examined using a fluorescence microscope (Zeiss 880, Germany and Leica SP5, Germany). The percentage of the positive area and cell number relative to DAPI was calculated by using ImageJ Software.

In Vivo Statistical Analysis: For the statistical analysis of data, GraphPad Prism (version 5.01, GraphPad Software) was used, and Image J software was used for the quantification of data. Data are presented as mean±standard error of the mean (SEM). One-way analysis of variance (ANOVA) with the Tukey post-hoc test was used to assess effects of multiple treatments in in-vivo experiments, p-values <0.05 were considered statistically significant.

### Discussion

Addressing the aforementioned challenges, biomaterial-based methods (e.g., 3D-printed discs, stem cell-seeded scaffolds, and drug-loaded hydrogels) have shown tremendous therapeutic potential for treating fibrocartilaginous tissue diseases by providing mechanical support, sustainably releasing chondrogenic differentiation factors, and replacing the unfavorable ECM. Nonetheless, their therapeutic outcomes are frequently hampered by crosstalk among different inhibitory signaling pathways. One such complicated inhibitory signaling is produced from degeneration-associated reactive oxygen species (ROS) that cause continuous apoptosis, inflammation, and uncontrollable differentiation of endogenous stem cells during acute injury and chronic phases of degeneration. Although reactive oxygen species (ROS) linked with degeneration and injuries are a common therapeutic target for treatingtissue damage, most traditional biomaterials cannot provide long-term effective anti-ROS signaling countermeasures from a single treatment.

Biodegradable/injectable nanomaterial-embedded peptide hydrogels (NEPH) are assembled from 1D dynamic peptide nanofibrils and 2D ROS-scavenging MnO₂ nanosheets for treating fibrocartilaginous tissue degeneration. In this Example, the nano-hybrid peptide hydrogel is applied to treat IVD, a disease that causes severe back pain and limited mobility and affects hundreds of thousands of patients worldwide. Remarkably, NEP hydrogels not only protect NP cells from apoptosis by efficiently scavenging ROS, the nano-hybrid (MnO₂) peptide hydrogel simultaneously allows for magnetic resonance imaging (MRI)-mediated monitoring and is mechanically versatile, with tunable stiffness through modulation of the ratio between organic (peptide) and inorganic (MnO₂ nanosheets) nanomaterials. Moreover, the NEP hydrogels can be 3D-printed into shapes matching injured tissue and modulate hydrogel drug-loaded release. The drug (growth factor differentiation 5 (GDF-5)) is further loaded onto the NEPH, which was subsequently injected into injured intervertebral discs, resulting in considerable regeneration of NP tissue, including critical ECM components like aggrecan and collagen, and enhanced cartilage regeneration.Furthermore, pain biomarkers were reduced, suggesting a potential behavioral improvement in pain perception reduction.

### Generating Nanomaterial-Embedded Peptide Hydrogel Through Hierarchical Self-Assembly

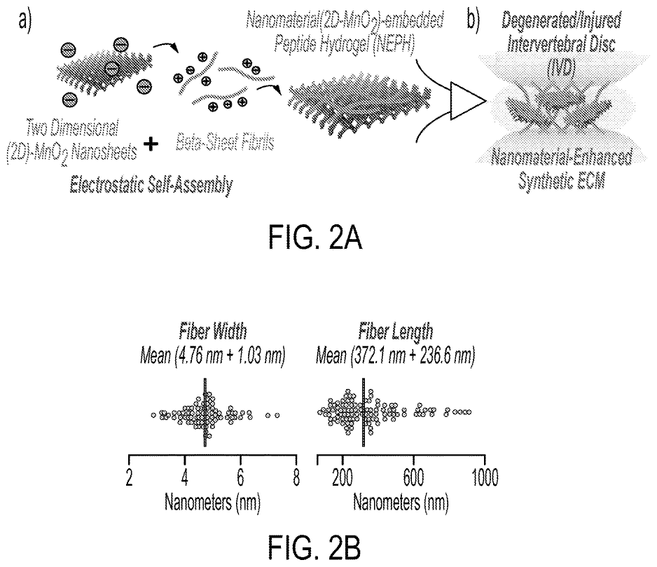

The extracellular matrix (ECM) environment predominantly comprises proteins, such as collagen, which self-assemble into one-dimensional fibril-like structures. Biomaterials, such as self-assembling synthetic peptides, have been utilized to recapitulate natural ECM by forming secondary structures such as alpha helices or β-sheets, which can induce gelation above threshold concentrations. Additionally, nanomaterials can be integrated within self-assembling peptides to form hybrid materials of a unique hierarchal structure. In prior work, integrating graphene oxide nanosheets with an octapeptide sequence (FEFKFEFK, SEQ ID NO: 1) was described, demonstrating the material's injectable capabilities and documenting a proliferative effect on IVD cell activity. However, graphene oxide nanosheets, a widely used nanomaterial, have unclear degradation methods and may not be suitable for long-term in vivo delivery. On the other hand, 2D-MnO₂ nanosheets are completely biodegradable through a redox reaction with naturally cell-secreted reductants such as ascorbic acid and glutathione. In this Example 2D-MnO₂ nanosheets and octapeptides were combined to develop a biodegradable hybrid injectable hydrogel through hierarchical self-assembly, which heirachly mimics natural ECM while simultaneously providing ROS-scavenging capabilities (FIG. 2). Alternating hydrophobic (e.g., phenylalanine (F)) and hydrophilic (lysine (K) and glutamate (E)] residues drove assembly through structural packing in aqueous solutions (FIG. 2A).

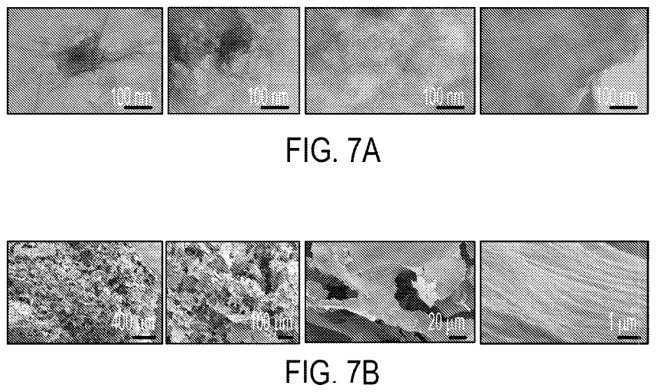

Transmission electron microscopy (TEM) showed individual peptide fibers ranging in lengths (~1-10 μm) and widths (~5-20 nm) (FIG. 7). At a peptide concentration of 5 mg/ml, the average fibril width was approximately 4.7 nm (+/−1.0 nm), while fibril length was approximately 372 nm (+/−237 nm) (FIG. 2B). It is worth noting that these dimensions closely align with natural collagen, which may be broken down into monomeric species and then assembled into tropocollagen, fibrils, and supramolecular assemblies. Introducing synthesized 2D-MnO₂ nanosheets through drop-casting and vigorous vortexing in FEFKFEFK (SEQ ID NO: 1) peptide aqueous solutions to form nanomaterial (MnO₂)-embedded peptide hydrogels (NEPHs) results in dense networks of individual nanosheets and peptide fibrils (FIG. 7). Fibril density increases in regions of 2D-MnO₂ nanosheets, likely attributable to prevailing electrostatic interactions between polar oxygen functional groups on 2D-MnO₂ nanosheets and hydrophilic lysine and glutamate residues. This increase in fibril density may promote cellular attachment within in vivo microenvironments, of which both 2D-MnO₂ nanosheets and peptide fibrils have been reported to promote. In addition, cellular penetration and migration within in vivo microenvironments may be promoted as scanning electron microscopy (SEM) images of lyophilized nanomaterial (MnO₂)-embedded peptide hydrogels (NEPHs) show porous hybrid structures of stacked 2D-MnO₂ nanosheets and peptide fibrils (FIG. 7). Data from atomic force microscopy (AFM) and confocal microscopy both confirm nanotopographical features and porous structures, respectively. Moreover, control over macroscale assembly of nanomaterials is desirable to precisely design biomaterials to treat IVDD and other central nervous system injuries and disorders. For this purpose, NEP hydrogels were successfully 3D-printed into numerous patterns of varying sizes and dimensions. However, these hydrogels can also be printed in disc-mimicking structures.

### Tailoring Mechanical, Biochemical, and Drug Release Properties of NEPHs for IVD Regeneration

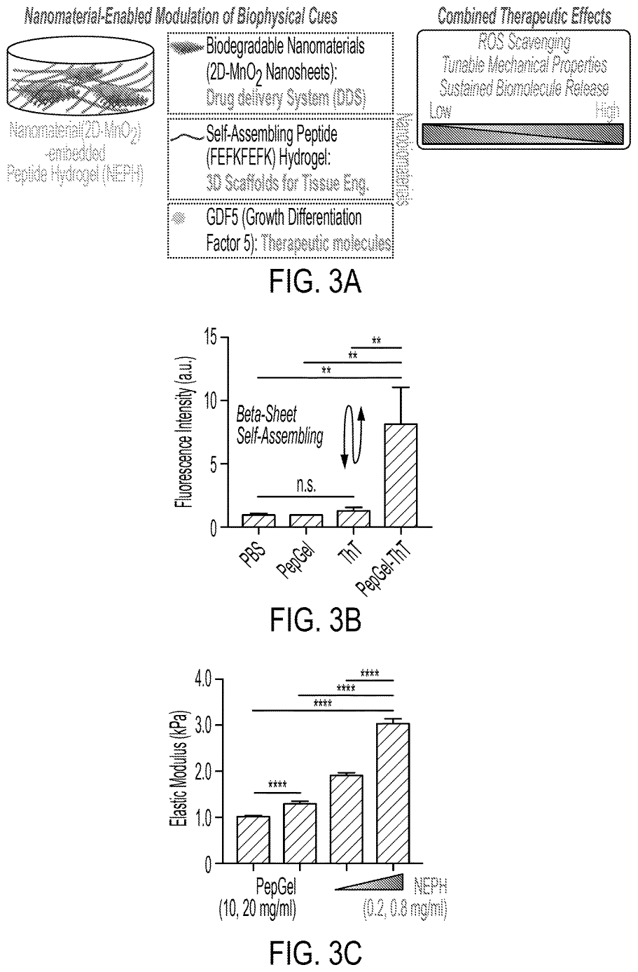

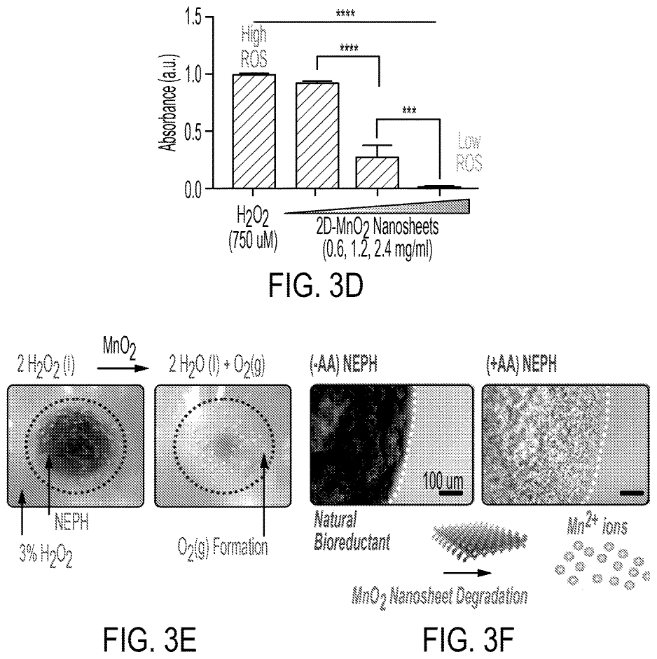

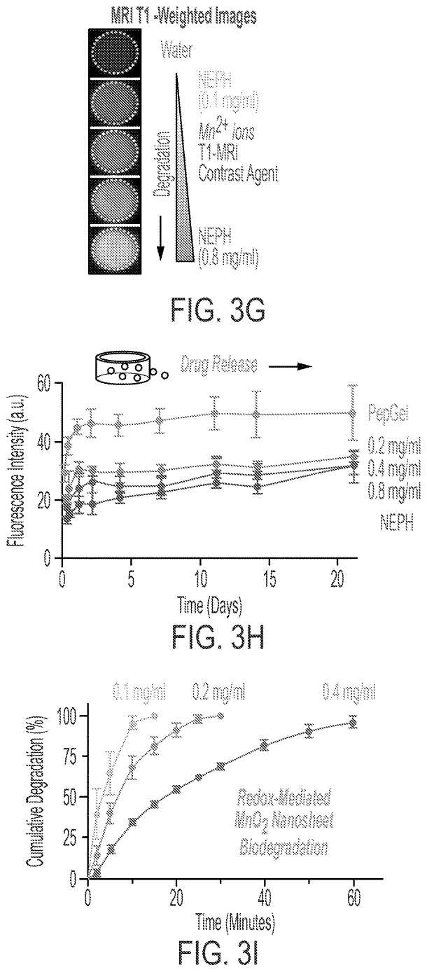

The nanomaterial (MnO₂)-embedded peptide hydrogels (NEPHs) as described herein were designed that incorporate 2D-manganese dioxide nanosheets to address these limitations while retaining ECM-mimicking properties of synthetic fiber-forming peptides (FIGS. 3A-3I). First, FEFKFEFK (SEQ ID NO: 1) peptides were confirmed to self-assemble to form secondary beta-sheet structures (FIG. 3B). Next, the effect of 2D-MnO₂ nanosheets on nanomaterial (MnO₂)-embedded peptide hydrogel (NEPH) stiffness was investigated. The nucleus pulposus region of the intervertebral disc is gelatinous, with high-water content, and abundant in type II collagen. Reports suggest the elastic modulus of the nucleus pulposus region is approximately 6 kPa. The nanomaterial (MnO₂)-embedded peptide hydrogels (NEPHs) of the present invention are tunable within a 1-3 kilopascal (kPa) range by tuning the concentration of nanosheets (FIG. 3C).

Reactive oxygen species, such as hydrogen peroxide (H₂O₂), are highly secreted during inflammation. Such reactive oxygen species can make intervertebral degeneration become worse by oxidizing proteins and cellular membranes, which can also lead to the death of cells. As a result of vascular failure, IVD degeneration can also create a hypoxic microenvironment, restricting nutritional and metabolic diffusion. NEP hydrogels with 2D-MnO₂ nanosheets can simultaneously provide ROS scavenging while producing oxygen as a byproduct.

Various concentrations of 2D-MnO₂ nanosheets were incubated in a solution of hydrogen peroxide (0.3%) and performed as a catalyst to accelerate the decomposition of hydrogen peroxide. A peroxide detection assay was then used to detect the presence of hydrogen peroxide after 2D-MnO₂ treatment and demonstrated increased ROS-scavenging, whereby a greater absorbance indicates a higher concentration of hydrogen peroxide (FIG. 3D). Oxygen gas formation from hydrogen peroxide decomposition can be visualized after incubation with nanomaterial (MnO₂)-embedded peptide hydrogels (NEPHs), which could potentially act as a source of oxygen and help alleviate hypoxic microenvironments during injury or degeneration FIG. 3E.

Moreover, as previously mentioned, certain hybrid nanomaterials containing graphene or carbon nanotubes have unique advantages, such as high drug loading but may not be fully biodegradable. 2D-MnO₂ nanosheets can be fully degraded via redox reaction through naturally secreted reductants, such as ascorbic acid (vitamin C) or glutathione (FIG. 3F). As a result, NEPHs are fully degradable via i) a redox mechanism for degradation of 2D-MnO₂ nanosheets and ii) proteolytic degradation of synthetic peptides. Notably, the degradation rate can be tuned through the concentration of 2D-MnO₂ nanosheets within nanomaterial (MnO₂)-embedded peptide hydrogels (NEPHs) (FIG. 3I). Besides, the rates at which drugs are released from biomaterials can be adjusted to optimize the release of therapeutics for improved treatment of acute or chronic inflammatory states. Degenerative conditions such as IVD may require a more sustained release of drugs and growth factors to combat chronic inflammation and cell death.

However, many conventional biomaterials, i.e., gelatin and alginate, and polymers, i.e., polycaprolactone (PCL) and polylactic-co-glycolic acid (PLGA), exhibit burst-release kinetics of encapsulated biomolecules. To evaluate the high drug loading and rate of growth factor release, a model growth factor, fluorescein isothiocyanate-conjugated insulin (FITC-insulin, 0.2 mg/ml), was loaded in NEPHs. Compared to hydrogels with no 2D-MnO₂ nanosheets (i.e. PepGel), NEP hydrogels with 2D-MnO₂ nanosheets showed a more sustained release of FITC-insulin, providing the potential to treat injuries requiring sustainable, localized drug release as opposed to global intravenous administration of biomolecules which rapidly diffuse and have short-lived effects (FIG. 3H).

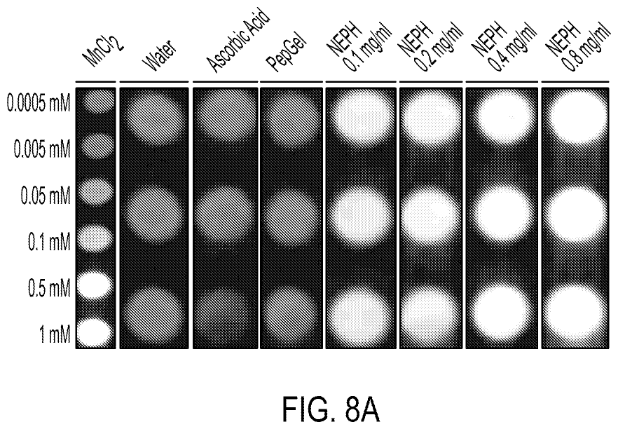

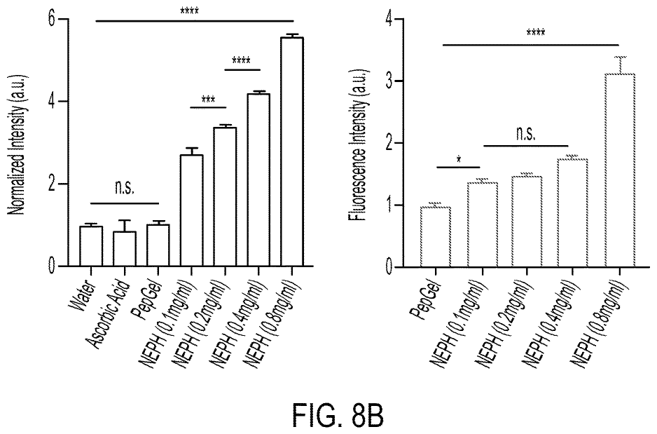

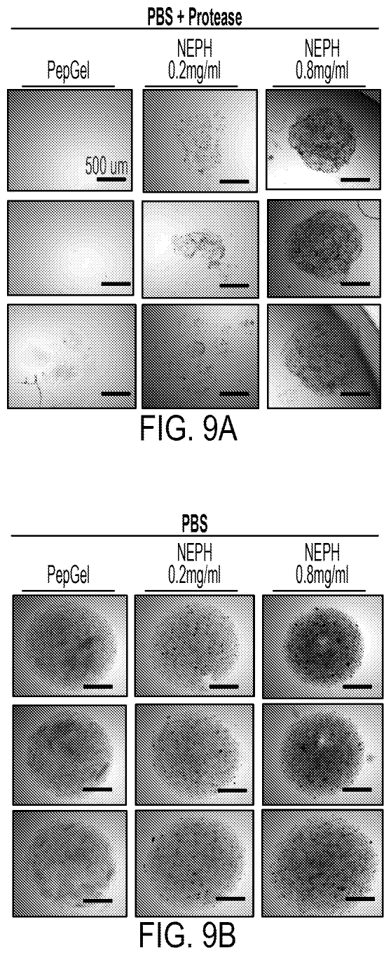

Finally, providing a detectable chemical signal to potentially monitor in vivo drug release and biomaterial degradation is a challenge to address in the biomaterial and nanomaterial fields. Fluorescence-based probes are limited because of the low penetrance of visible light. On the other hand, MRI has been widely employed as a non-invasive in vivo imaging method and can be utilized to monitor biomaterial degradation. Uniquely, NEP hydrogels is characterized as MRI contrast agent. Fully degraded NEP hydrogels (via ascorbic acid treatment) loaded with FITC-insulin release Mn²⁺ ion contrast agent and FITC-insulin in a concentration-dependent manner, providing the advantage of correlating drug release with biomaterial degradation (FIGS. 3G and 8A-3B).

### Enhancing Disc Cell Survival by NEPH In Vitro

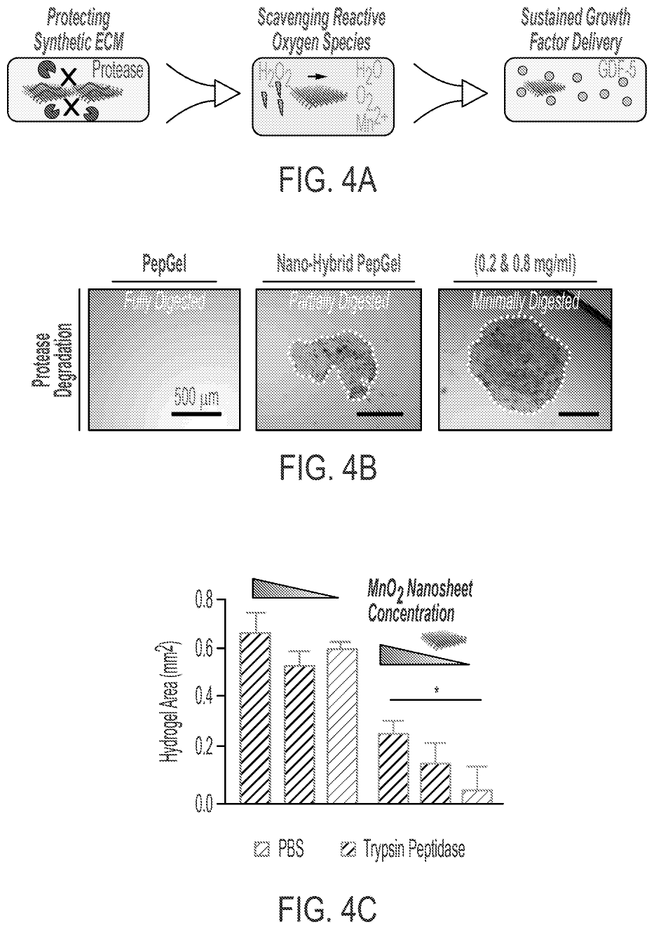

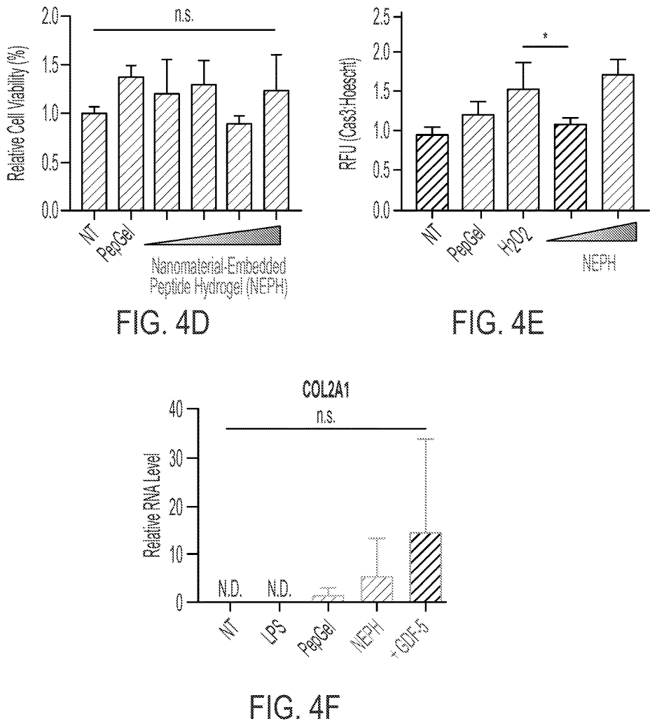

In an effort to elucidate potential mechanisms of disc repair with the treatment of nanomaterial (MnO₂)-embedded peptide hydrogel (NEPH) s in vivo, NEPH biological properties were evaluated in vitro (FIGS. 4A-4F). First, as previously reported, a major limitation of synthetic peptide-derived ECM hydrogels is their susceptibility to rapid proteolytic degradation (FIG. 4A). While fully biodegradable materials are advantageous, the rapid degradation of synthetic ECM may hinder tissue regeneration and overall injury repair over the long-term. To this end, EP hydrogels were prepared and the rate of proteolytic degradation was investigated in the presence of a serine protease, trypsin, which reportedly cleaves at lysine and arginine residues.

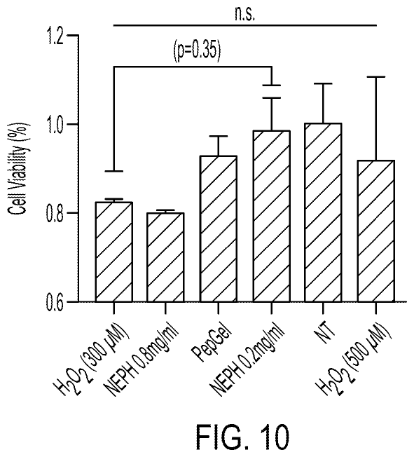

NEP hydrogels were incubated in trypsin solution (10 mg/ml) overnight, and hydrogel size was quantified (FIG. 4B and FIGS. 4C, 9). Matrix metalloproteinases (MMPs) are heavily secreted during severe degeneration and injury. As a consequence, NEPHs with increasing 2D-MnO2 nanosheet delayed proteolytic degradation. This can be used to tune the biomaterial degradation rate and retain the integrity of synthetic ECM in vivo. Next, the ROS-scavenging effect of NEP hydrogels in a biological system was investigated in vitro using primary human nucleus pulposus cells (hNPCs) (FIG. 4D). First, the cell viability of NEP hydrogels were investigated with increasing concentrations of 2D-MnO2 nanosheets (FIG. 4G). Next, NEPHs (0.2 mg/ml, 0.8 mg/ml MnO₂ nanosheet concentration) were incubated in a solution of H₂O₂ (0.3%) and mixed for approximately five minutes to scavenge reactive oxygen species and decompose H₂O₂ to water and oxygen gas. Next, the solution was added to hNPC cell culture media and cultured overnight. As a result, the cell viability presto blue assay revealed that cell survival was higher in NEPH with relatively lower concentrations (0.2 mg/ml) compared to those treated with solely 300 μM hydrogen peroxide (FIG. 10). Oddly, 500 μM treatment of hydrogen peroxide alone to hNPCs showed a wide range of cell viability (FIG. 10). Furthermore, higher MnO₂ nanosheet concentrations (0.8 mg/ml) were observed to trigger an adverse cellular response; therefore, subsequent in vivo studies in NEPHs used a MnO₂ nanosheet concentration of 0.1 mg/ml to avoid severe side effects.

Immunostaining for apoptosis marker cleaved caspase-3 (Cas3) demonstrated a similar trend to cell viability where Cas3 to DAPI ratio, normalized to the non-treated control, was lowest in both non-treated and NEPHs with a 0.2 mg/ml concentration of 2D-MnO₂ nanosheets embedded within (FIGS. 4E, 11, and 12). These results imply that the scavenging of hydrogen peroxide species (i.e., ROS) from NEPHs may protect hNPCs from elevated expression caspase and undergoing apoptosis. Last, it has been reported that growth factors can stimulate IVD cells to produce ECM proteins and promote cellular proliferation. To this end, delivery of growth differentiation factor 5 (GDF-5)-loaded NEPHs was investigated using a transwell cell culture model. Here, GDF-5-loaded NEPHs were inserted into the top chamber while hNPCs were cultured in the bottom chamber. After stimulating hNPCs to an inflammatory state by administering lipopolysaccharide (LPS, 10 ug/ml), hydrogels were added to the top chamber and cultured overnight.

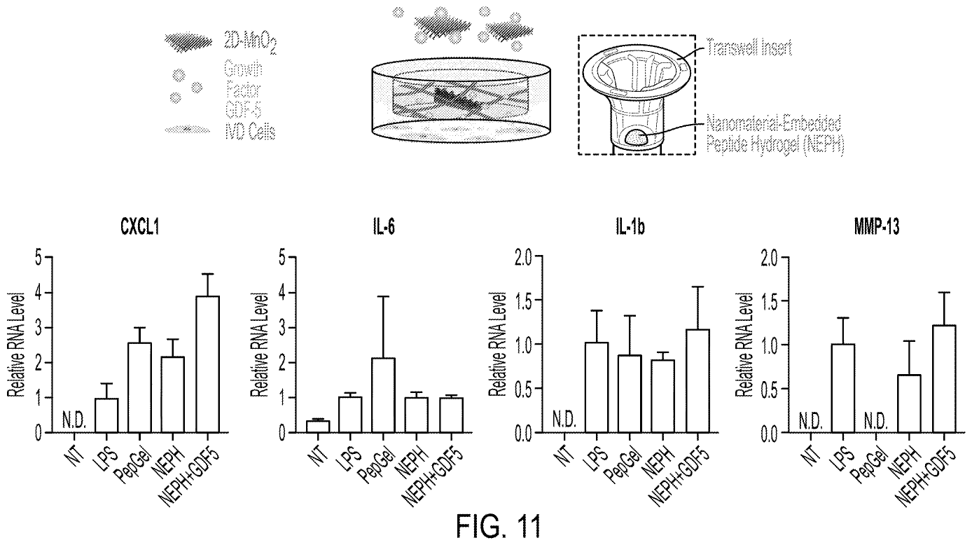

Gene expression of collagen II (COL2A1) was then investigated with quantitative polymerase chain reaction and showed that GDF-5 loaded NEPHs had the highest COL2A1 expression, followed by NEPH without GDF-5 and PepGel alone (FIG. 41). Pro-inflammatory cytokine levels (CXCL1, IL-6, IL-1B, and MMP-13) were also investigated FIG. 11. This promising result may be due to the fact that i) GDF-5 has been reported to stimulate collagen 2 expression, and ii) the octapeptide sequence in NEPHs (FEFKFEFK, SEQ ID NO: 1), had previously been reported to stimulate IVD cell proliferation. Furthermore, Mn²⁺ ion has been linked to enhanced collagen expression by acting as an essential metal co-factor in the enzyme prolidase, which is responsible for producing the amino acid proline.

### Enhancing Disc Tissue Regeneration by NEPH In Vivo

After confirming the therapeutic ROS-scavenging effects of nanomaterial (MnO2)-embedded peptide hydrogel (NEPH) s with 2D-MnO₂ nanosheets and the potential anti-inflammatory ECM-stimulating delivery of GDF-5, the in vivo therapeutic potential in a rat nucleotomy model was investigated by removing the NP. This experiment was designed and performed to better understand how GDF-5 could be used to treat inflammatory disorders and and aid tissue regeneration by establishing an ECM-mimicking microenvironment, scavenge ROS after nucleotomy, and stimulate cellular proliferation and ECM synthesis.

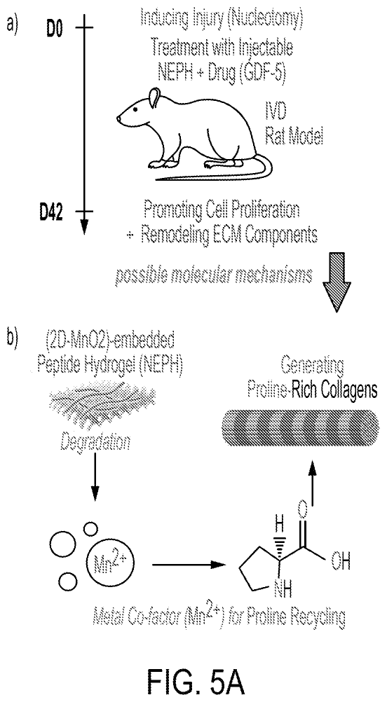

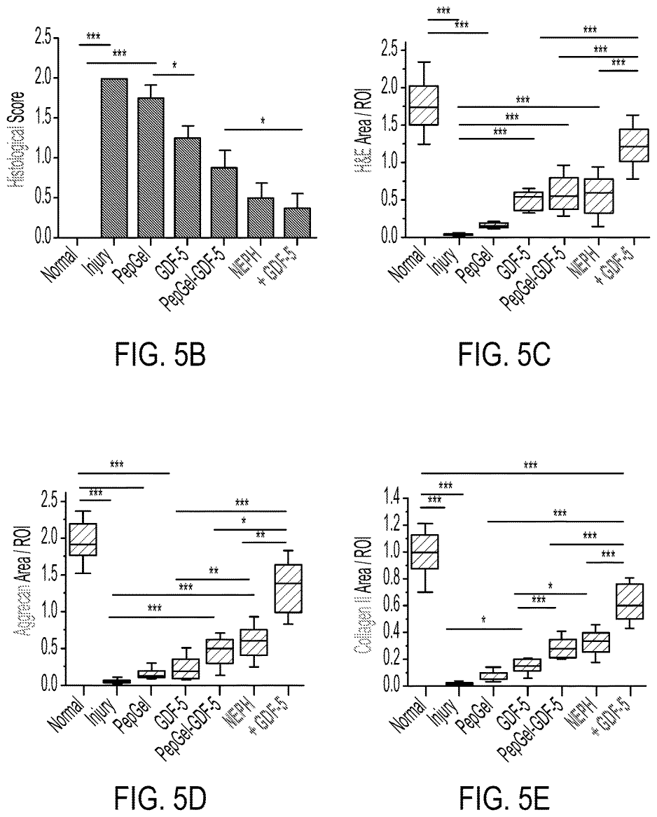

To this end, a nucleotomy (removal of the nucleus pulposus region of the intervertebral disc) was performed on Sprague-Dawley rats, and 10 ml of NEP hydrogels loaded with GDF-5 (10 μg/ml) were immediately injected into the injury site along with NEP hydrogels without GDF-5, PepGel alone, GDF-5 saline solution, and PepGel-GDF-5 (FIG. 5A). Rats were sacrificed at week 6 (day 42) and analyzed to investigate tissue regeneration within the NP, AF, and endplate regions.

Hematoxylin and eosin (H&E) staining of the injury site at 6 weeks showed NEPHs with 2D-MnO₂ nanosheets loaded with GDF-5 regenerated distinct AF and NP regions dense with cellular nuclei and ECM components, yielding the lowest histological score of all experimental groups. Compared to all the other experimental groups, the distance between adjacent endplates in rats injected with NEPH or NEPH with GDF-5 was much greater. Moreover, it has been well established that the NP region contains chondrocyte-like cells that readily express and secrete ECM proteins such as collagen II (COL2) and essential proteoglycans such as aggrecan (ACAN) to support the gelatinous and flexible structure of the intervertebral disc. Thus, immunohistochemistry (IHC) in the NP region shows the highest expression for COL2 in the NEPHs loaded with GDF-5 condition, thereby restoring a favorable ECM microenvironment.

There have been reports that suggest the high water content and gel-like characteristic of the NP region are mediated by the expression of aggrecan from NP cells. Here, aggrecan levels were highly elevated in NEPHs loaded with GDF-5 compared to all other experimental groups. Peptide and saline groups showed minimal aggrecan expression after six weeks. Moreover, GDF-5 alone showed decreased aggrecan expression, which could be attributed to the short-term therapeutic effects of a burst release of growth factors. Interestingly, NEPHs without GDF-5 loaded on 2D-MnO₂ nanosheets also demonstrated increased aggrecan expression compared to peptide and GDF-5 alone groups. Again, this may be due to the therapeutic effects of Mn²⁺ ions, a byproduct of NEPH degradation.

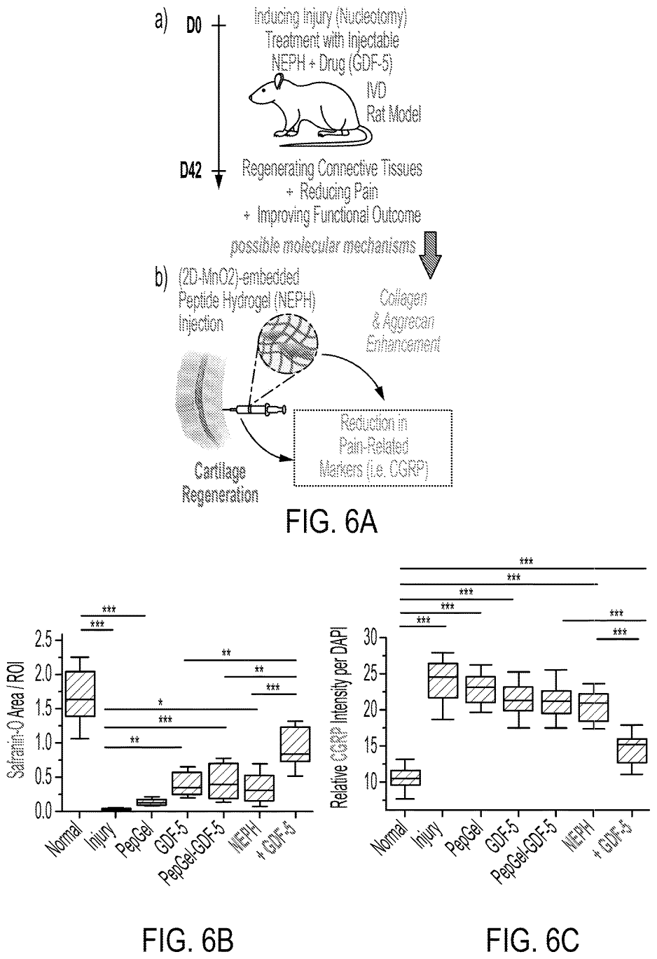

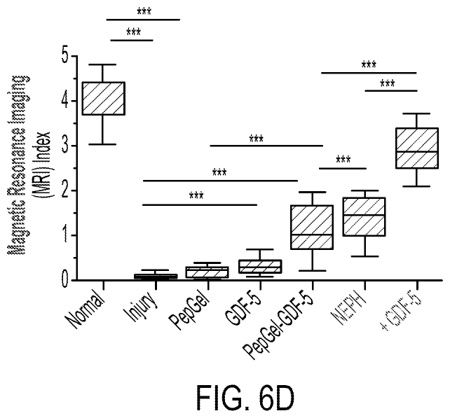

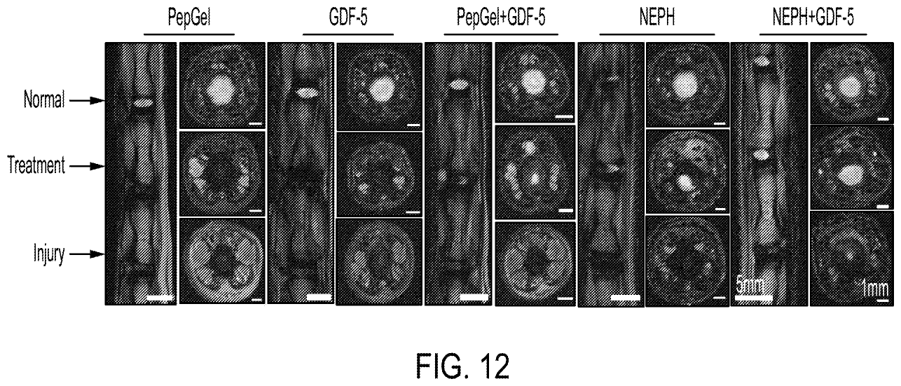

Next, IVD regeneration and pain alleviation were investigated (FIG. 6A). Moreover, non-invasive T2-weighted MRI analysis confirms that NEPH with GDF-5 restored high water content to the degenerated IVD region (FIG. 12). Given that peptide hydrogels displayed limited T2-weighted MRI signal, this result is most likely related to tissue regeneration rather than hydrogel water retention. Indeed, following the regeneration of collagen and aggrecan because of NEPH with GDF-5, enhanced ECM formation in degenerated IVD was also shown (FIG. 6A).

Improving Functional Outcome and Reducing Pain after Disc Degeneration by NEPH

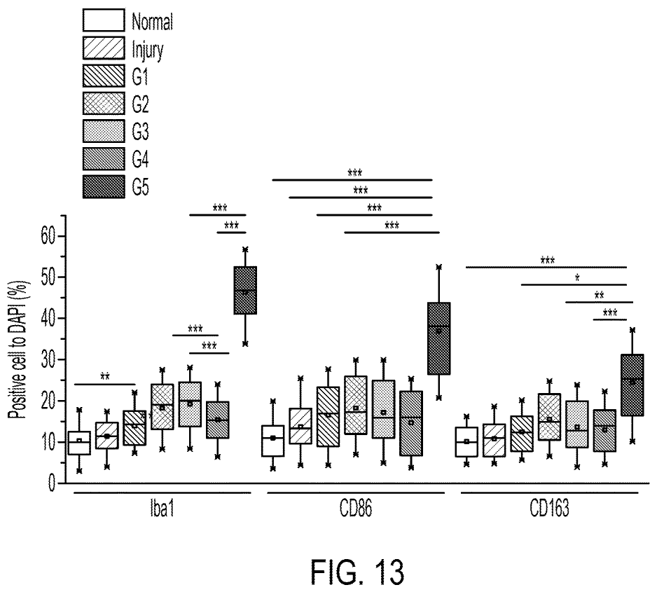

Lastly, tissue regeneration, accompanied by an overall reduction in pain, would be the most desired therapeutic outcome. Previously, it has been reported that even moderate cases of IVDD cause chronic back pain, decreasing quality of life. Therefore, the expression of Calcitonin gene-related peptide (CGRP), a known biomarker of perceived pain in nociceptive pathways in the nervous system was investigated. Six-weeks post-injection, CGRP levels in NEPHs with GDF-5 were drastically reduced compared to injured discs and all other conditions, suggesting a reduction in pain sensation (FIG. 6C). An analysis of the inflammation six weeks post-injection of NEPHs with GDF-5 showed an increase in both M1 (pro-inflammatory) and M2 (anti-inflammatory) macrophage phenotypes, where elevated levels of Iba1, CD86, and CD163 were observed (FIG. 13). Further, pain evaluation by Von Frey test was performed from week 2 (day 14) to week 6 (day 42). To this end, the 50% withdrawal threshold value exhibited a general trend from week 2 to week 6, where NEPHs with GDF-5 outperformed all other experimental conditions. At week 5 and week 6, withdrawal threshold values of NEPHs with GDF-5 were statistically significant (p<0.05) compared to peptide hydrogels alone. Therefore, nanomaterial embedded peptide hydrogels successfully loaded with therapeutic growth factors reduced pain-related biomarker CGRP, which likely translated into a reduction in the Von Frey evaluation and improved functional outcomes.

In summary, nanomaterial (MnO₂)-embedded peptide hydrogels (NEPHs) provide a versatile hybrid nanomaterial designed to stimulate regeneration in the intervertebral disc (IVD). NEPHs have unique hierarchal properties that mimic natural collagen ECM at the nanoscale and microscale, while providing 3D-printing macroscale capabilities over conventional polymeric hydrogel materials used for biomedical applications such as gelatin or alginate. NEPHs have superior biophysical features due to nanomaterial-mediated accelerated drug release, tunable biodegradation, ROS scavenging, and MRI activity. Furthermore, the addition of growth differentiation factor 5 (GDF-5) in NEPHs promoted ECM-stimulating effect in vitro and in vivo. NEPHs loaded with GDF-5 were injected into a nucleotomy rat model to regenerate lost tissue in the AF and NP regions. After 6 weeks, increased ECM regeneration (i.e., collagen and aggrecan), cellular proliferation, and T2-MRI signal intensity compared to both PepGel hydrogels (with no embedded nanomaterials) and growth factors alone. Histological analysis at 6 weeks shows IVD regeneration with NEPHs, while behavioral analysis shows a positive trend. Overall, NEPHs repaired the degenerated IVD after nucleotomy and restored a healthy ECM environment to permit NF and AF regeneration.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A hydrogel composite composition, comprising:
nanosheets comprising manganese dioxide (MnO₂);
self-assembling peptides; and
a biologically active material.

2. The hydrogel composite composition of claim 1, wherein the mean size of the nanosheets ranges from about 10 nm to about 500 nm along the longest dimension of the nanosheets.

3. The hydrogel composite composition of claim 1, wherein the self-assembling peptides comprise a peptide having an amino acid sequence of SEQ ID NO: 1, or a salt, a hydrate or an isomer thereof.

4. The hydrogel composite composition of claim 1, wherein a ratio of the nanosheets to the self-assembling peptides ranges from 1:10 to 20:1.

5. The hydrogel composite composition of claim 1, wherein the self-assembling peptides are embedded in the nanosheets.

6. The hydrogel composite composition of claim 1, wherein the biologically active material comprises a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, psychoactive compound, or combinations thereof.

7. The hydrogel composite composition of claim 5, wherein the biologically active material comprises a growth factor.

8. The hydrogel composite composition of claim 6, wherein the biologically active material comprises GDF5.

9. The hydrogel composite composition of claim 7, wherein a concentration of the growth factor ranges from 1 to 100 μg/ml.

10. The hydrogel composite composition of claim 1, further comprising a matrix comprising a solvent component and a polymer resin.

11. The hydrogel composite composition of claim 1, wherein the hydrogel composition has a water content of about 10 to 80% by weight.

12. A method of treating a tissue injury in a subject comprising:
administering a hydrogel composite composition of claim 1 to the subject.

13. The method of claim 12, wherein the subject has been or is suffering from an injury in a neural tissue or a nervous system, a cartilage, a muscle, or a bone.

14. The method of claim 13, wherein the subject has been or is suffering from an injury in a neural tissue or a nervous system.

15. The method of claim 14, wherein the subject has been or is suffering from degenerated intervertebral discs (IVD).

16. The method of claim 14, wherein the subject has been or is treated with neurosurgery.

17. (canceled)

18. (canceled)

19. (canceled)

20. (canceled)

21. A method of preparing a hydrogel composite, comprising:
preparing self-assembling peptides from an aqueous solution comprising peptide precursors by rapid mixing;
preparing an admixture comprising (i) nanosheets comprising manganese dioxide (MnO₂) and (ii) the self-assembling peptide precursors; and
adding a biological active material to the admixture.

22. (canceled)

23. A method of monitoring treatment of a tissue injury in a subject comprising:
administering a hydrogel composite composition of claim 1 to the subject; and
detecting the Mn²⁺ released from the nanosheets.

24. (canceled)

25. A kit for treating a tissue in a subject, comprising:
a hydrogel composite composition of claim 1; and
an applicator.

26. (canceled)

27. A contrast agent for magnetic resonance imaging (MRI) comprising a hydrogel composite composition of claim 1.
